(19) United States
(12) Patent Application Publication
(10) Pub. No.: US 2021/0393481 A1
(43) Pub. Date: Dec. 23, 2021

(54) SYSTEMS AND METHODS FOR PRESERVATIVE REMOVAL FROM OPHTHALMIC FORMULATIONS COMPRISING COMPLEXING AGENTS

(71) Applicant: TearClear Corp., Copley, OH (US)

(72) Inventors: Michael T. MALANGA, Midland, MI (US); Howard L. GOLUB, Copley, OH (US)

(21) Appl. No.: 17/463,051

(22) Filed: Aug. 31, 2021

Related U.S. Application Data
(63) Continuation of application No. 16/782,628, filed on Feb. 5, 2020.
(60) Provisional application No. 62/941,398, filed on Nov. 27, 2019.
(60) Provisional application No. 62/802,132, filed on Feb. 6, 2019.

(51) Int. Cl. A61J 1/14; A61K 9/00; B01D 39/16; A61K 38/13; A61K 47/40; A61K 31/5575; A61K 31/573; A61K 47/18

(52) U.S. Cl. CPC A61J 1/1443; A61J 1/1468; A61K 9/0048; B01D 39/1676; A61K 47/18; A61K 47/40; A61K 31/5575; A61K 31/573; A61K 38/13

(57) ABSTRACT

Systems and methods for removing a preservative from a solution, emulsion, or suspension may include an ophthalmic agent, a complexing agent, and a matrix. A method for administering an ophthalmic agent may include: providing a solution, emulsion, or suspension comprising a hydrophobic ophthalmic agent, a preservative, and a complexing agent, wherein the complexing agent is configured to host the hydrophobic ophthalmic agent; and providing a polymeric matrix, wherein the complexing agent is configured to reduce an affinity of the ophthalmic agent for the polymeric matrix and wherein the polymeric matrix is configured to selectively absorb the preservative when the solution, emulsion, or suspension is passed therethrough.

### CROSS REFERENCE TO RELATED APPLICATIONS

This application is a continuation of U.S. patent application Ser. No. 16/782,628, filed Feb. 5, 2020, which claims the benefit of U.S. Provisional Application No. 62/802,132, filed Feb. 6, 2019, and U.S. Provisional Application No. 62/941,398, filed Nov. 27, 2019, both of which are incorporated by reference in the disclosure of this application.

### BACKGROUND

The present disclosure generally relates to systems and methods for removal of preservatives and removing a preservative from a fluid comprising an ophthalmic agent.

Prior approaches to the removal of a preservative from a fluid comprising an ophthalmic agent prior to administration to an eye may be less than ideal in at least some respects. Patients suffering from chronic diseases may use daily eye drop instillations, for example for the treatment of glaucoma. In order to prevent bacterial growth, commercially available eye drop formulations typically use a preservative, in order to address possible bacterial contamination.

The potential for ocular damage from the preservatives may be elevated among patients suffering from chronic diseases which may require daily eye drop instillations for periods of years to decades, such as glaucoma patients. Potential toxic side effects from preservative-free eye drops can be lower than from their preserved counterparts. Patients using preserved eye drops and experiencing toxicity symptoms, such as allergy, blepharitis, or dry eye, may show improvement upon switching to preservative-free formulations.

Although preservative removal devices have been proposed, the prior approaches can be less than ideal and overly complex, in at least some instances. For example, some prior approaches can remove more therapeutic agent than would be ideal, for example, in an effort to produce “preservative-free” eye drops. Other prior approaches may absorb the ophthalmic agent over time resulting in varying dosage as a function time, which may reduce the shelf life of the eye drop formulation.

### SUMMARY

The present disclosure relates to systems and methods for removing a preservative from a solution, emulsion, or suspension comprising an ophthalmic agent. In light of the above, there is a clear unmet need for improved systems and methods for removing a preservative from a fluid comprising an ophthalmic agent and a preservative. One technical problem to be solved in meeting this unmet need is the ability to selectively remove the preservative without changing the concentration of the therapeutically effective ophthalmic agent in the fluid. In some cases, the interaction between the ophthalmic agent and a preservative removing device may be tuned by the addition of a complexing agent. In some cases, an ophthalmic agent may be sufficiently soluble without a complexing agent. Ideally these systems and methods would address at least some of the above drawbacks of prior approaches and reduce patient exposure to preservative while maintaining consistent dosage.

In an aspect, a method for administering an ophthalmic agent is provided. The method may comprise: providing a solution, emulsion, or suspension comprising a hydrophobic ophthalmic agent, a preservative, and a complexing agent, wherein the complexing agent is configured to host the hydrophobic ophthalmic agent; and providing a polymeric matrix, wherein the complexing agent is configured to reduce an affinity of the ophthalmic agent for the polymeric matrix and wherein the polymeric matrix is configured to selectively absorb the preservative when the solution, emulsion, or suspension is passed therethrough.

In some embodiments, the complexing agent and the hydrophobic ophthalmic agent form an inclusion compound. In some embodiments, the complexing agent comprises a cyclodextrin. In some embodiments, the cyclodextrin is sized to host the hydrophobic ophthalmic agent within a hydrophobic interior of the cyclodextrin. In some embodiments, the cyclodextrin is at least one of (2-Hydroxypropyl)-α-cyclodextrin, (2-Hydroxypropyl)-β-cyclodextrin, (2-Hydroxypropyl)-γ-cyclodextrin, α-cyclodextrin, β-cyclodextrin, γ-cyclodextrin, methyl-α-cyclodextrin, methyl-β-cyclodextrin, methyl-γ-cyclodextrin, dimethyl-beta-cyclodextrin, highly sulphated-beta-cyclodextrin, 6-monodeoxy-6-N-mono(3-hydroxy)propylamino-beta-cyclodextrin, or a randomly or selectively substituted alpha, beta or gamma cyclodextrin.

In some embodiments, a concentration of the complexing agent is less than 200 micromolar. In some embodiments, a concentration of the complexing agent is greater than the concentration of the ophthalmic agent by about 10:1 by mole to about 200:1 by mole. In some embodiments, a concentration of the complexing agent is greater than the concentration of the ophthalmic agent by at least 2 percent by mole. In some embodiments, the complexing agent is a micelle forming surfactant.

In some embodiments, the hydrophobic ophthalmic agent comprises latanoprost, bimatoprost, dexamethasone, cyclosporine or travoprost, or any prostaglandin analog drug. In some embodiments, the concentration of the ophthalmic agent is less than 200 millimolar. In some embodiments, the concentration of the ophthalmic agent is less than 0.05% by weight. In some embodiments, the preservative is benzalkonium chloride. In some embodiments, the concentration of the preservative is less than 0.05% by weight.

In some embodiments, wherein the polymeric matrix is a polymeric hydrogel. In some embodiments, the polymeric matrix comprises 2-hydroxyethylmethacrylate. In some embodiments, the polymeric matrix comprises tert-butyl methacrylate. In some embodiments, the polymeric matrix comprises a crosslinker. In some embodiments, the crosslinker is SR-9035.

In some embodiments, the solution, emulsion, or suspension is disposed within a chamber of a compressible bottle. In some embodiments, the polymeric matrix is disposed between the chamber and an outlet of a compressible bottle. In some embodiments, compression of the compressible bottle passes the solution, emulsion, or suspension through the polymeric matrix to the outlet. In some embodiments, compression of the compressible bottle forms a drop at the outlet. In some embodiments, the concentration of the ophthalmic agent after passing though the polymeric matrix is at least 80% of a concentration of the ophthalmic agent before passing through the polymeric matrix. In some embodiments, the concentration of the ophthalmic agent after passing though the polymeric matrix is at least 90% of a concentration of the ophthalmic agent before passing through the polymeric matrix. In some embodiments, the concentration of the ophthalmic agent after passing though the polymeric matrix is at least 95% of the concentration of the ophthalmic agent before passing through the polymeric matrix. In some embodiments, the concentration of the preservative after passing though the polymeric matrix is less than 10% of the concentration of the preservative before passing through the polymeric matrix. In some embodiments, the concentration of the preservative after passing though the polymeric matrix is less than 5% of the concentration of the preservative before passing through the polymeric matrix. In some embodiments, the concentration of the preservative after passing though the polymeric matrix is less than 1% of the concentration of the preservative before passing through the polymeric matrix. In some embodiments, a timescale for drop formation is less than 3 seconds.

In some embodiments, the molar ratio of the ophthalmic agent to the complexing agent in the solution, emulsion, or suspension is about 200:about 1, about 175:about 1, about 150:about 1, about 125:about 1, about 100:about 1, about 75:about 1, about 50:about 1, about 25:about 1, about 10:about 1, about 9.5:about 1, about 9.0:about 1, about 8.5:about 1, about 8.0:about 1, about 7.5:about 1, about 7.0:about 1, about 6.5:about 1, about 6.0:about 1, about 5.5:about 1, about 5.0:about 1, about 4.5:about 1, about 4.0:about 1, about 3.5:about 1, about 3.0:about 1, about 2.5:about 1, about 2.0:about 1, about 1.9:about 1, about 1.8:about 1, about 1.7:about 1, about 1.6:about 1, about 1.5:about 1, about 1.4:about 1, about 1.3:about 1, about 1.2:about 1, about 1.19:about 1, about 1.18:about 1, about 1.17:about 1, about 1.16:about 1, about 1.15:about 1, about 1.14:about 1, about 1.13:about 1, about 1.12:about 1, or about 1.11:about 1.

In some embodiments, the polymeric matrix is polyvinyl alcohol crosslinked with citric acid or other suitable crosslinking agent to render it a hydrogel. In some embodiments, the polymeric matrix is selected from crosslinked polyvinylpyrrolidone, crosslinked polyethylene oxide, crosslinked polyacrylamides, crosslinked copolymers of methacrylic acid, polyacrylic acid, or copolymers selected from poly (acrylic acid-co-acrylamide), or poly (methacrylic acid-co-acrylamide). In some embodiments, the polymeric matrix is hydrogel prepared from polyacrylamide crosslinked with at least one crosslinking monomer selected from N,N′-methylenebis(acrylamide) (MBAM), triacrylamido triazine (TATZ), SR 351, or SR9035; and the crosslinked polyacrylamide is modified with at least one modifying monomer selected from methyl methacrylate (MAA), 2-acrylamido-2-methylpropane sulfonic acid (AMPS), 2-sulfoethyl methacrylate (SEM), acrylic acid (AA), or vinylphosphonic acid (VP).

In some embodiments, the polymeric matrix is hydrogel prepared from polyacrylamide crosslinked with N,N′-methylenebis(acrylamide) (MBAM); and the crosslinked polyacrylamide is modified with 2-sulfoethyl methacrylate (SEM). In some embodiments, the polymeric matrix is hydrogel prepared from polyacrylamide crosslinked with at least one crosslinking monomer selected from N,N′-methylenebis(acrylamide) (MBAM), triacrylamido triazine (TATZ), SR 351, or SR9035; the crosslinked polyacrylamide material is isolated; and the crosslinked polyacrylamide material is modified with at least one modifying monomer selected from methyl methacrylate (MAA), 2-acrylamido-2-methylpropane sulfonic acid (AMPS), 2-sulfoethyl methacrylate (SEM), acrylic acid (AA), or vinylphosphonic acid (VP).

In some embodiments, the polymeric matrix is hydrogel prepared from polyacrylamide crosslinked with N,N′-methylenebis(acrylamide) (MBAM; the crosslinked polyacrylamide material is isolated; and the crosslinked polyacrylamide material is modified with at least one modifying monomer selected from 2-acrylamido-2-methylpropane sulfonic acid (AMPS), or 2-sulfoethyl methacrylate (SEM). In some embodiments, the crosslinked polyacrylamide material is isolated in the form of spherical beads.

In another aspect, a method for administering an ophthalmic agent is provided. The method may comprise: applying pressure to a compressible bottle comprising: a solution, emulsion, or suspension comprising a hydrophobic ophthalmic agent, a preservative, and a complexing agent, wherein the complexing agent is configured to host the hydrophobic ophthalmic agent; wherein the complexing agent is configured to reduce an affinity of the ophthalmic agent for the polymeric matrix; and wherein the polymeric matrix is configured to selectively absorb the preservative when the solution, emulsion, or suspension is passed therethrough.

In another aspect, a preservative removing device is provided. The device may comprise: a solution, emulsion, or suspension comprising a hydrophobic ophthalmic agent, a preservative, and a complexing agent, wherein the complexing agent is configured to host the hydrophobic ophthalmic agent; wherein the complexing agent is configured to reduce an affinity of the ophthalmic agent for the polymeric matrix; and wherein the polymeric matrix is configured to selectively absorb the preservative when the solution, emulsion, or suspension is passed therethrough.

In some embodiments, the complexing agent and the hydrophobic ophthalmic agent form an inclusion compound. In some embodiments, the complexing agent comprises a cyclodextrin. In some embodiments, the cyclodextrin is sized to host the hydrophobic ophthalmic agent within a hydrophobic interior of the cyclodextrin. In some embodiments, the cyclodextrin is at least one of (2-Hydroxypropyl)-α-cyclodextrin, (2-Hydroxypropyl)-β-cyclodextrin, (2-Hydroxypropyl)-γ-cyclodextrin, α-cyclodextrin, β-cyclodextrin, γ-cyclodextrin, methyl-α-cyclodextrin, methyl-β-cyclodextrin, or methyl-γ-cyclodextrin. In some embodiments, a concentration of the complexing agent is less than 200 micromolar. In some embodiments, a concentration of the complexing agent is greater than a concentration of the ophthalmic agent by about 10:1 by mole. In some embodiments, a concentration of the complexing agent is greater than a concentration of the ophthalmic agent by at least 2 percent by mole. In some embodiments, the complexing agent is a micelle forming surfactant.

In some embodiments, the hydrophobic ophthalmic agent comprises latanoprost, bimatoprost, dexamethasone, cyclosporine, travoprost, or any prostaglandin analog drug. In some embodiments, the concentration of the ophthalmic agent is less than 200 millimolar. In some embodiments, the concentration of the ophthalmic agent is less than 0.05% by weight. In some embodiments, the preservative is benzalkonium chloride. In some embodiments, the concentration of the preservative is less than 0.05% by weight.

In some embodiments, the polymeric matrix is a hydrogel. In some embodiments, the polymeric matrix comprises 2-hydroxyethylmethacrylate. In some embodiments, the polymeric matrix comprises tert-butyl methacrylate. In some embodiments, the polymeric matrix comprises a crosslinker. In some embodiments, the crosslinker is SR-9035.

In some embodiments, the solution, emulsion, or suspension is disposed within a chamber of a compressible bottle. In some embodiments, the polymeric matrix is disposed between the chamber and an outlet of a compressible bottle. In some embodiments, compression of the compressible bottle passes the solution, emulsion, or suspension through the polymeric matrix to the outlet. In some embodiments, compression of the compressible bottle forms a drop at the outlet. In some embodiments, the concentration of the ophthalmic agent after passing though the polymeric matrix is at least 80% of a concentration of the ophthalmic agent before passing through the polymeric matrix. In some embodiments, the concentration of the ophthalmic agent after passing though the polymeric matrix is at least 90% of the concentration of the ophthalmic agent before passing through the polymeric matrix. In some embodiments, the concentration of the ophthalmic agent after passing though the polymeric matrix is at least 95% of the concentration of the ophthalmic agent before passing through the polymeric matrix. In some embodiments, the concentration of the preservative after passing though the polymeric matrix is less than 10% of the concentration of the preservative before passing through the polymeric matrix. In some embodiments, the concentration of the preservative after passing though the polymeric matrix is less than 5% of the concentration of the preservative before passing through the polymeric matrix. In some embodiments, the concentration of the preservative after passing though the polymeric matrix is less than 1% of the concentration of the preservative before passing through the polymeric matrix. In some embodiments, a timescale for drop formation is less than 3 seconds.

In some embodiments, the polymeric matrix is polyvinyl alcohol crosslinked with citric acid or other suitable crosslinking agent to render it a hydrogel. In some embodiments, the polymeric matrix is selected from crosslinked polyvinylpyrrolidone, crosslinked polyethylene oxide, crosslinked polyacrylamides, crosslinked copolymers of methacrylic acid, polyacrylic acid, or copolymers selected from poly (acrylic acid-co-acrylamide), or poly (methacrylic acid-co-acrylamide). In some embodiments, the polymeric matrix is hydrogel prepared from polyacrylamide crosslinked with at least one crosslinking monomer selected from N,N′-methylenebis(acrylamide) (MBAM), triacrylamido triazine (TATZ), SR 351, or SR9035; and the crosslinked polyacrylamide is modified with at least one modifying monomer selected from methyl methacrylate (MAA), 2-acrylamido-2-methylpropane sulfonic acid (AMPS), 2-sulfoethyl methacrylate (SEM), acrylic acid (AA), or vinylphosphonic acid (VP). In some embodiments, the polymeric matrix is hydrogel prepared from polyacrylamide crosslinked with N,N′-methylenebis(acrylamide) (MBAM); and the crosslinked polyacrylamide is modified with 2-sulfoethyl methacrylate (SEM).

In some embodiments, the polymeric matrix is hydrogel prepared from polyacrylamide crosslinked with at least one crosslinking monomer selected from N,N′-methylenebis(acrylamide) (MBAM), triacrylamido triazine (TATZ), SR 351, or SR9035; the crosslinked polyacrylamide material is isolated; and the crosslinked polyacrylamide material is modified with at least one modifying monomer selected from methyl methacrylate (MAA), 2-acrylamido-2-methylpropane sulfonic acid (AMPS), 2-sulfoethyl methacrylate (SEM), acrylic acid (AA), or vinylphosphonic acid (VP). In some embodiments, the polymeric matrix is hydrogel prepared from polyacrylamide crosslinked with N,N′-methylenebis(acrylamide) (MBAM; the crosslinked polyacrylamide material is isolated; and the crosslinked polyacrylamide material is modified with at least one modifying monomer selected from 2-acrylamido-2-methylpropane sulfonic acid (AMPS), or 2-sulfoethyl methacrylate (SEM). In some embodiments, the crosslinked polyacrylamide material is isolated in the form of spherical beads.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates a system for providing an ophthalmic agent, in accordance with some embodiments;

FIG. 2A illustrates an eye drop bottle comprising a matrix in a removable cap, in accordance with some embodiments;

FIG. 2B illustrates a compressible bottle comprising a matrix, in accordance with some embodiments;

FIG. 2C illustrates a compressible bottle comprising a matrix in the neck of a nozzle, in accordance with some embodiments;

FIG. 3 is a flow chart of a method of delivering an ophthalmic agent, in accordance with some embodiments.

FIG. 4A illustrates a guest-host interaction of a complexing agent and an ophthalmic agent of the present disclosure, in accordance with some embodiments;

FIG. 4B illustrates a guest-host interaction of a cyclodextrin and Latanoprost, in accordance with some embodiments;

FIG. 5 illustrates a micelle and an ophthalmic agent of the present disclosure, in accordance with some embodiments; and

FIG. 6 illustrates an example SEM image of hydrogel D-322-056-02-AW.

### DETAILED DESCRIPTION

Unless defined otherwise, all technical and scientific terms used herein have the same meaning as is commonly understood by one of skill in the art to which this invention belongs. All patents and publications referred to herein are incorporated by reference.

As used in the specification and claims, the singular form “a”, “an” and “the” includes plural references unless the context clearly dictates otherwise.

As used herein, and unless otherwise specified, the term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 30%, 25%, 20%, 15%, 10%, 9%, 8%, 7%, 6%, 5%, 4%, 3%, 2%, 1%, 0.5%, 0.1%, or 0.05% of a given value or range. In certain embodiments, the term “about” or “approximately” means within 40.0 mm, 30.0 mm, 20.0 mm, 10.0 mm 5.0 mm 1.0 mm, 0.9 mm, 0.8 mm, 0.7 mm, 0.6 mm, 0.5 mm, 0.4 mm, 0.3 mm, 0.2 mm or 0.1 mm of a given value or range.

As used herein, the terms “comprises,” “comprising,” or any other variation thereof, are intended to cover a nonexclusive inclusion, such that a process, method, article, or apparatus that comprises a list of elements does not include only those elements but may include other elements not expressly listed or inherent to such process, method, article, or apparatus.

As used herein, the terms “user”, “subject” or “patient” are used interchangeably. As used herein, the terms “subject” and “subjects” refers to an animal (e.g., birds, reptiles, and mammals), a mammal including a primate (e.g., a monkey, chimpanzee, and a human) and a non-primate (e.g., a camel, donkey, zebra, cow, pig, horse, cat, dog, rat, and mouse). In certain embodiments, the mammal is 0 to 6 months old, 6 to 12 months old, 1 to 5 years old, 5 to 10 years old, 10 to 15 years old, 15 to 20 years old, 20 to 25 years old, 25 to 30 years old, 30 to 35 years old, 35 to 40 years old, 40 to 45 years old, 45 to 50 years old, 50 to 55 years old, 55 to 60 years old, 60 to 65 years old, 65 to 70 years old, 70 to 75 years old, 75 to 80 years old, 80 to 85 years old, 85 to 90 years old, 90 to 95 years old or 95 to 100. In some embodiments, the subject or patient is a pig. In certain embodiments, the pig is 0 to 6 months old, 6 to 12 months old, 1 to 5 years old, 5 to 10 years old or 10 to 15 years old. The natural lifespan of a pig is 10-15 years.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms may be based on objective or subjective parameters; including the results of a physical examination, neuropsychiatric exams, and/or a psychiatric evaluation. The term “treating” and conjugations thereof, include prevention of an injury, pathology, condition, or disease.

In some embodiments, the term “prevent” or “preventing” as related to a disease or disorder may refer to a compound that, in a statistical sample, reduces the occurrence of the disorder or condition in the treated sample relative to an untreated control sample, or delays the onset or reduces the severity of one or more symptoms of the disorder or condition relative to the untreated control sample.

An “effective amount” is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of a “therapeutically effective amount” is an amount sufficient to contribute to the treatment, prevention, or reduction of a symptom or symptoms of a disease, which could also be referred to as a “therapeutically effective amount.” A “reduction of” a symptom or symptoms (and grammatical equivalents of this phrase) means decreasing of the severity or frequency of the symptom(s), or elimination of the symptom(s). The exact amounts may depend on the purpose of the treatment and may be ascertainable by one skilled in the art using known techniques.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “substituted” refers to moieties having substituents replacing a hydrogen on one or more carbons or heteroatoms of the structure. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms.

Embodiments of the present disclosure provide a preservative removing device. The preservative removing device may comprise (1) a solution, emulsion, or suspension comprising a hydrophobic ophthalmic agent, a preservative, and a complexing agent, wherein the complexing agent is configured to host the hydrophobic ophthalmic agent; wherein the complexing agent is configured to reduce an affinity of the ophthalmic agent for the polymeric matrix; and (2) a polymeric matrix, wherein the polymeric matrix is configured to selectively absorb the preservative when the solution, emulsion, or suspension is passed therethrough.

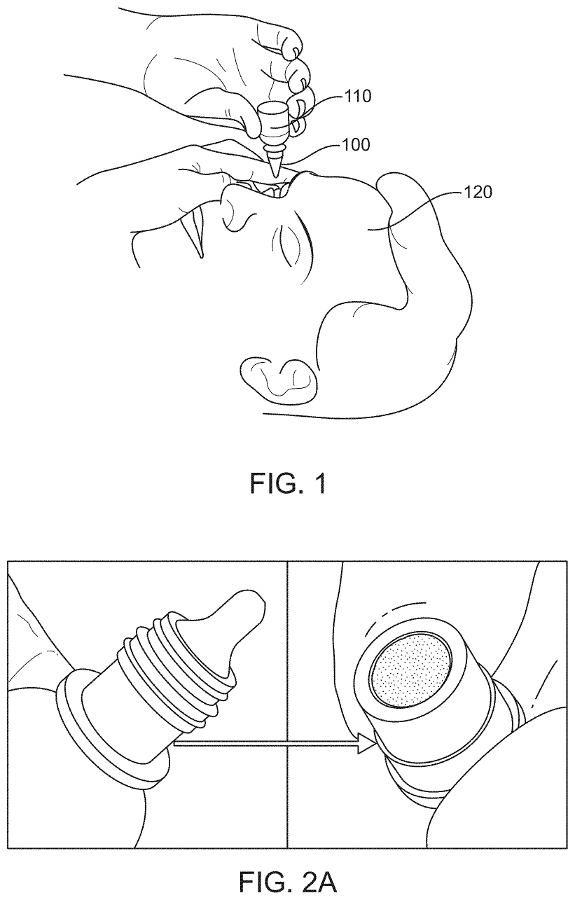

FIG. 1 illustrates a system for providing an ophthalmic agent, in accordance with some embodiments. The system may comprise a preservative removing device 100 disposed within a neck of a compressible bottle 110. A pressure may be applied by a user 120 (e.g. a patient, a subject) to compressible bottle 110 to pass a solution, emulsion, or suspension through a preservative removing device to thereby deliver an ophthalmic agent to an eye.

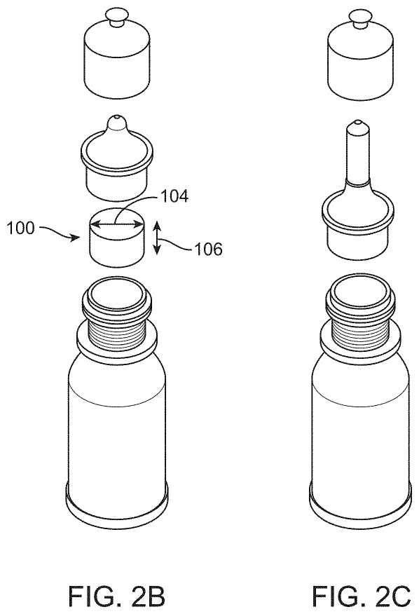

FIG. 2A illustrates an eye drop bottle comprising a matrix in a removable cap, in accordance with some embodiments. FIG. 2B illustrates a compressible bottle comprising a matrix, in accordance with some embodiments. FIG. 2C illustrates a compressible bottle comprising a matrix in the neck of a nozzle, in accordance with some embodiments. A porous preservative removing device may be situated in the neck of the eye drop bottle leading to the drop exit. In some embodiments, the matrix may be situated in a section of the tip of the eye drop bottle. A tip may be included in the bottle to allow a matrix to be positioned therein. The preservative removing device can be separate filter that is attached to the formulation dispensing unit through a suitable connector for use. The preservative removing device may comprise a portion of a multi-dosing device for delivery of an ophthalmic solution. A multi-dosing device may comprise a compressible bottle that has an outlet extension containing the preservative removing device. When the hydrophilic polymeric gel is dry, it may have dimensions smaller than the internal dimensions of the outlet extension but may have dimensions larger than the internal dimensions of the outlet extension when swollen with the ophthalmic solution. A preservative removing device may self-support within the compressible bottle. A preservative removing device may be press fit into the bottle. A preservative removing device may be held within a secondary container (e.g. a sachet) within the compressible bottle.

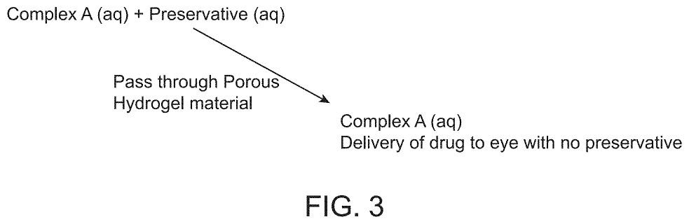

FIG. 3 is a flow chart of a method of delivering an ophthalmic agent, in accordance with some embodiments. Disclosed herein are methods for administering an ophthalmic agent. A method of administering an ophthalmic agent may comprise: providing solution, emulsion, or suspension comprising a hydrophobic ophthalmic agent, a preservative, and a complexing agent, wherein the complexing agent is configured to host the hydrophobic ophthalmic agent; passing the solution, emulsion or suspension through a preservative removing device; and delivering the ophthalmic agent to an eye.

A method of administering an ophthalmic agent may comprise: providing a solution, emulsion, or suspension comprising a hydrophobic ophthalmic agent, a preservative, and a complexing agent, wherein the complexing agent is configured to host the hydrophobic ophthalmic agent; and providing a polymeric matrix, wherein the complexing agent is configured to reduce an affinity of the ophthalmic agent for the polymeric matrix and wherein the polymeric matrix is configured to selectively absorb the preservative when the solution, emulsion, or suspension is passed therethrough.

A method for administering an ophthalmic agent may comprise: applying pressure to a compressible bottle comprising: a solution, emulsion, or suspension comprising a hydrophobic ophthalmic agent, a preservative, and a complexing agent, wherein the complexing agent is configured to host the hydrophobic ophthalmic agent; wherein the complexing agent is configured to reduce an affinity of the ophthalmic agent for the polymeric matrix; and wherein the polymeric matrix is configured to selectively absorb the preservative when the solution, emulsion, or suspension is passed therethrough.

### Solution, Emulsion, or Suspension

Provided herein are ophthalmic formulations comprising an ophthalmic agent, a complexing agent, and a preservative. In some embodiments, ophthalmic formulations provided herein are solutions, emulsions, and/or suspensions of an ophthalmic agent, a complexing agent, and a preservative. In some embodiments, provided herein are compositions comprising a therapeutically effective amount of any ophthalmic therapeutic compound, or salt of any one of the preservatives, ophthalmic agents, and/or complexing agents of the present disclosure. In some embodiments, a solution, emulsion, or suspension may be used in any of the methods described herein. The solution, emulsion, or suspension may additionally comprise one or more pharmaceutically acceptable excipients.

In some embodiments, a composition of complexing agent, therapeutic agent, and/or a preservative may be used for the treatment of a therapeutic disorder such as, dry eye, bacterial infection, glaucoma, hypertension, inflammation, allergic conjunctivitis, hypotrichosis of the eyelashes, fungal infection, etc. Additionally, or alternatively, a composition of a preservative, therapeutic agent, and/or a complexing agent may be used during a preventative, diagnostic, or therapeutic ophthalmological procedure, for example, local anesthetic, pupil dilation, etc. A solution, emulsion, or suspension administered to the eye may be administered topically, for example, with an eye drop. In some embodiments, the compounds, or salts thereof, of the disclosure with low aqueous solubility may be formulated as aqueous suspensions.

### Ophthalmic Agent

Embodiments of the present disclosure may provide an ophthalmic agent for delivery to an eye. The ophthalmic agent may be a therapeutic agent. The therapeutic agent may comprise one or more ophthalmic agents. In some embodiments, the disclosure provides solutions, emulsions, or suspensions of a preservative, a complexing agent, and an ophthalmic agent. In some embodiments, the solutions, emulsions, or suspension may comprise a preservative removal agent, (e.g. in embodiments where the preservative removal agent may comprise a portion of a solution, emulsion, or suspension comprising an ophthalmic agent and a preservative). In other embodiments, the preservative removal agent may be separate from the solution, emulsion, or suspension comprising the ophthalmic agent, the complexing agent, and the preservative (e.g. in embodiments where the preservative removal agent may be located within the neck of a bottle). Ophthalmic agents may comprise compounds and salts, for use in the treatment of ophthalmic diseases. Optionally, in any embodiment, the solution, emulsion, or suspension may additionally comprise one or more pharmaceutically acceptable excipients. The disclosed compounds and salts can be used, for example, for the treatment or prevention of vision disorders and/or for use during ophthalmological procedures for the prevention and/or treatment of ophthalmic disorders. The flowing list of examples is not intended to be limiting.

An ophthalmic agent may be integrated into a fluid, which may flow from a container to an eye through an outlet of a compressible bottle. In some embodiments, the fluid may comprise a solution, emulsion, or suspension comprising an ophthalmic agent. The solution, emulsion, or suspension may comprise the ophthalmic agent. Example ophthalmic agents which may be used in conjunction with a compressible bottle include but are not limited to: timolol, dorzolamide, dexamethasone phosphate, dexamethasone, Betimol, olopatadine, brimonidine, tetrahydrozoline, latanoprostene bunod, latanoprost, bimatoprost, travoprost and combinations of any two or more thereof. Ophthalmic agents may comprise brand name drugs and formulations including, but not limited to, Timoptic, Xalatan, Combigan, Lumigan, Pataday, Pazeo, Trusopt, Cosopt, Alphagan, Visine, Vyzulta, Vesneo, and other agents described herein such as in the following tables. The ophthalmic agents may be dissolved in aqueous solution. The solution may be sterilized and buffered to appropriate pH. In some embodiments, the solution may comprise inactive ingredients such as sodium chloride, sodium citrate, hydroxyethyl cellulose, sodium phosphate, citric acid, sodium dihydrogen phosphate, polyoxyl 40 hydrogenated castor oil, tromethamine, boric acid, mannitol, glycerine edetate disodium, sodium hydroxide, and/or hydrochloric acid. In some embodiments, the fluid comprises a preservative in addition to an ophthalmic agent. Example preservatives include but are not limited to: benzalkonium chloride (BAK), alcohols, parabens, methyl paraben, polyparaben, EDTA, chlorhexidine, quaternary ammonium compounds, Purite®, stabilized oxychloro complexes, Sofzia®, sorbic acid, Sodium perborate, polyquaternium-1, chlorobutanol, cetrimonium chloride, edetate disodium, etc.

In some embodiments the ophthalmic agent is latanoprost. In some embodiments the ophthalmic agent is bimatoprost. In some embodiments the ophthalmic agent is travoprost. In some embodiments the ophthalmic agent is latanoprost and the preservative is benzalkonium chloride (BAK). In some embodiments the ophthalmic agent is bimatoprost and the preservative is benzalkonium chloride (BAK). In some embodiments the ophthalmic agent is travoprost and the preservative is benzalkonium chloride (BAK).

Ophthalmic agents for the treatment of, for example, dry eye, bacterial infection, glaucoma, hypertension, inflammation, allergic conjunctivitis, hypotrichosis of the eyelashes, fungal infection, etc. and ophthalmic agents used for local anesthetic, pupil dilation, etc. may be administered to a patient as a solution, emulsion, or suspension delivered to an eye topically via a compressible bottle, a dropper bottle, or similar delivery mechanism. The solution, emulsion, or suspension may be subject to contamination such as microbial, fungal, or particulate contamination, which may be adverse to patient health. In order to prevent such contamination a preservative may be added to the solution, emulsion, or suspension; however, patient exposure to preservatives may have adverse effects to eye health. It may be advantageous to limit patient exposure to preservative by providing a preservative removing device which may remove a preservative from the solution, emulsion, or suspension.

In some embodiments, the ophthalmic agent to be dispensed comprises an active ingredient selected from cyclosporine and lifitegrast. In such embodiments, the ophthalmic agent may be an active ingredient in the treatment of dry eye.

In some embodiments, the ophthalmic agent to be dispensed comprises an active ingredient selected from sulfacetamide sodium, ofloxacin, gatifloxacin, ciprofloxacin, moxifloxacin, tobramycin, levofloxacin, prednisolone acetate, polymyxin B sulfate, and trimethoprim. In some embodiments, the ophthalmological formulation to be dispensed comprises the active ingredients sulfacetamide sodium and prednisolone acetate. In some embodiments, the ophthalmological formulation to be dispensed comprises the active ingredients polymyxin B sulfate and trimethoprim. In such embodiments, the ophthalmic agent may be an active ingredient in the treatment of a bacterial infection.

In some embodiments, the ophthalmic agent to be dispensed comprises an active ingredient selected from brimonidine tartrate, bimatoprost, levobunolol hydrochloride, brinzolamide, betaxolol hydrochloride, pilocarpine hydrochloride, apraclonidine, travoprost, timolol maleate, latanoprost, dorzolamide hydrochloride, timolol maleate, and tafluprost. In some embodiments, the ophthalmological formulation to be dispensed comprises the active ingredients brimonidine tartrate and timolol maleate. In some embodiments, the ophthalmological formulation to be dispensed comprises the active ingredients brinzolamide and brimonidine tartrate. In such embodiments, the ophthalmic agent may be an active ingredient in the treatment of glaucoma or hypertension.

In some embodiments, the ophthalmic agent to be dispensed comprises an active ingredient selected from ketorolac tromethamine, fluorometholone, prednisolone acetate, difluprednate, fluorometholone acetate, nepafenac, dexamethasone, diclofenac sodium, bromfenac, gentamicin, tobramycin, neomycin, and polymyxin B sulfate. In some embodiments, the ophthalmological formulation to be dispensed comprises the active ingredients gentamicin and prednisolone acetate. In some embodiments, the ophthalmological formulation to be dispensed comprises the active ingredients tobramycin and dexamethasone. In some embodiments, the ophthalmological formulation to be dispensed comprises the active ingredients neomycin, polymyxin B sulfate and dexamethasone. In such an embodiment, the ophthalmic agent may be an active ingredient in the treatment of inflammation.

In some embodiments, the ophthalmic agent to be dispensed comprises an active ingredient selected from nedocromil sodium, epinastine HCl, alcaftadine, lodoxamide tromethamine, emedastine difumarate, and olopatadine hydrochloride. In such embodiments, the ophthalmic agent may be an active ingredient in the treatment of allergic conjunctivitis.

In some embodiments, the ophthalmic agent to be dispensed comprises an active ingredient selected from proparacaine hydrochloride and tetracaine hydrochloride. In such embodiments, the ophthalmic agent may be a local anesthetic.

In some embodiments, the ophthalmic agent to be dispensed comprises an active ingredient selected from cyclopentolate hydrochloride, atropine sulfate, and tropicamide. In some embodiments, the ophthalmological formulation to be dispensed comprises the active ingredients cyclopentolate hydrochloride and phenylephrine hydrochloride. In such embodiments, the ophthalmic agent may dilate pupils.

In some embodiments, the ophthalmic agent to be dispensed comprises the active ingredient natamycin. In such embodiments, the ophthalmic agent may be an active ingredient in the treatment of fungal infection.

In some embodiments, the ophthalmic agent to be dispensed comprises an active ingredient selected from lipoic acid choline ester chloride, rebamipide, pilocarpine, ketorolac, aceclidine, tropicamide, sodium hyaluronate, diclofenac sodium, pilocarpine HCl, and ketorolac. In some embodiments, the ophthalmological formulation to be dispensed comprises the active ingredients aceclidine and tropicamide. In some embodiments, the ophthalmological formulation to be dispensed comprises the active ingredients sodium hyaluronate and diclofenac sodium and pilocarpine HCl. In some embodiments, the ophthalmological formulation to be dispensed comprises the active ingredients pilocarpine and ketorolac. In such embodiments, the ophthalmic agent may be an active ingredient in the treatment of presbyopia.

In some embodiments, solutions, emulsions, or suspensions of the disclosure comprise a compound or salt of any ophthalmic agent of the present disclosure, wherein the compound or salt of the ophthalmic agent is largely free of impurities, such as at least about 80 wt % pure, at least about 81% pure, at least about 82% pure, at least about 83% pure, at least about 84% pure, at least about 85% pure, at least about 86% pure, at least about 87% pure, at least about 88% pure, at least about 89% pure, at least about 90% pure, at least about 91% pure, at least about 92% pure, at least about 93% pure, at least about 94% pure, at least about 95% pure, at least about 96% pure, at least about 97% pure, at least about 98% pure, at least about 99% pure, at least about 99.1% pure, at least about 99.2% pure, at least about 99.3% pure, at least about 99.4% pure, at least about 99.5% pure, at least about 99.6% pure, at least about 99.7% pure, at least about 99.8% pure, or at least about 99.9% pure.

In some embodiments, solutions, emulsions, or suspensions of the disclosure comprise a compound or salt of any ophthalmic agent of the present disclosure, wherein the ophthalmic agent is about 70% to about 99.99%, about 80% to about 99.9%, about 85% to about 99%, about 90% to about 99%, about 95% to about 99%, about 97% to about 99%, about 98% to about 99%, about 98% to about 99.9%, about 99% to about 99.99%, about 99.5% to about 99.99%, about 99.6% to about 99.99%, about 99.8 to about 99.99%, or about 99.9% to about 99.99% free of impurities.

The amount of the compound or salt of the ophthalmic agent in a solution, emulation, or suspension of the present disclosure can be measured as a percentage of mass per volume. In some embodiments, a solution, emulsion, or suspension such as an aqueous solution of the disclosure, comprises from about 0.05 wt % to about 10 wt % of the compound or salt of any of the ophthalmic agents disclosed herein. In some embodiments, a solution, emulsion, or suspension such as an aqueous solution of the disclosure, comprises about 0.01 wt %, about 0.02 wt %, about 0.03 wt %, about 0.04 wt %, about 0.05 wt %, about 0.06 wt %, about 0.07 wt %, about 0.08 wt %, about 0.09 wt %, about 0.1 wt %, about 0.2 wt %, about 0.3 wt %, about 0.4 wt %, about 0.5 wt %, about 0.6 wt %, about 0.7 wt %, about 0.8 wt %, about 0.9 wt %, about 1 wt %, about 1.1 wt %, about 1.2 wt %, about 1.3 wt %, about 1.4 wt %, about 1.5 wt %, about 1.6 wt %, about 1.7 wt %, about 1.8 wt %, about 1.9 wt %, about 2 wt %, about 2.1 wt %, about 2.2 wt %, about 2.3 wt %, about 2.4 wt %, about 2.5 wt %, about 2.6 wt %, about 2.7 wt %, about 2.8 wt %, about 2.9 wt %, about 3 wt %, about 3.1 wt %, about 3.2 wt %, about 3.3 wt %, about 3.4 wt %, about 3.5 wt %, about 3.6 wt %, about 3.7 wt %, about 3.8 wt %, about 3.9 wt %, about 4 wt %, about 4.1 wt %, about 4.2 wt %, about 4.3 wt %, about 4.4 wt %, about 4.5 wt %, about 5 wt %, about 6 wt %, about 7 wt %, about 8 wt %, about 9 wt %, or about 10 wt % of a compound or salt of the ophthalmic agent described herein.

A compound or salt of the ophthalmic agent described herein can be present in a solution, emulsion, or suspension of the present disclosure at a concentration of, for example, about 500 nM, about 600 nM, about 700 nM, about 800 nM, about 900 nM, about 1 μM, about 2 μM, about 3 μM, about 4 μM, about 5 μM, about 6 μM, about 7 μM, about 8 μM, about 9 μM, about 10 μM, about 20 μM, about 30 μM, about 40 μM, about 50 μM, about 60 μM, about 70 μM, about 80 μM, about 90 μM, about 100 μM, about 150 μM, about 200 μM, about 250 μM, about 300 μM, about 350 μM, about 400 μM, about 450 μM, about 500 μM, about 550 μM, about 600 μM, about 650 μM, about 700 μM, about 750 μM, about 800 μM, about 850 μM, about 900 μM, about 1 mM, about 5 mM, about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, or about 100 mM. The compound of an ophthalmic agent described herein may be present in a solution, emulsion, or suspension within a range of concentrations, the range being defined by an upper and lower value selected from any of the preceding concentrations. For example, the compound or salt of an ophthalmic agent of the disclosure may be present in the solution, emulsion, or suspension at a concentration of from about 1 nM to about 100 mM, about 10 nM to about 10 mM, about 100 nM to about 1 mM, about 500 nM to about 1 mM, about 1 mM to about 50 mM, about 10 mM to about 40 mM, about 20 mM to about 35 mM, or about 20 mM to about 30 mM.

### Preservative

The present disclosure provides formulations comprising one or more preservatives for solutions, emulsions, or suspensions of ophthalmic agents of the present disclosure. Preservatives may comprise compounds and salts, for use as preservatives for solutions, emulsions, or suspensions of ophthalmic agents. The one or more preservatives may for example prevent microbial and/or fungal growth. The one or more preservatives may for example prevent physical or chemical deterioration of an ophthalmic agent.

Non-limiting examples of preservative agents include benzalkonium chloride, ethylenediaminetetraacetic acid (EDTA), chlorobutanol, phenylmercuric acetate, phenylmercuric nitrate, chlorhexidine acetate, thimerosal, benzethonium chloride, sorbic acid, alcohols, parabens (e.g., methylparaben, polyparaben), chlorhexidine, quaternary ammonium compounds, cetrimonium bromide, cetramide, cetyltrimethylammonium bromide, hexadecyltrimethylammonium bromide polyquaternium-1 (Polyquad®), stabilized oxychloro complexes (Purite®), solutions of borate, sorbitol, propylene glycol, and zinc (Sofzia®), sodium perborate (GenAqua®), cetrimonium chloride, edetate disodium, etc. In some embodiments, a formulation of the disclosure comprises the preservative of quaternary ammonium compounds. In some embodiments the preservative is benzalkonium chloride (BAK).

In some embodiments, the particulate plug may further include a preservative removing compound or a preservative deactivating compound. Preservative removing or deactivating compounds can decrease toxicity of a formulation to be delivered through typical separation methods including, but not limited to, adsorption, ion exchange, chemical precipitation, or solvent extraction. Preservative removing or deactivating compounds can include, but are not limited to, activated charcoal, antioxidants, ethylenediaminetetraacetic acid (EDTA), anionic hydrogels, cationic compounds, neutralizing agents, or combinations thereof.

The Purite® preservative system includes Stabilized Oxychloro Complex (SOC), a combination of chlorine dioxide, chlorite, and chlorate. When exposed to light, SOC dissociates into water, oxygen, sodium, and chlorine free radicals which cause oxidation of intracellular lipids and glutathione, interrupting vital enzymes for cell function and maintenance. For preservatives such as Purite® which produce chlorine free radicals, the particulate plug of the disclosure can include a material that has a high affinity for free radicals such as activated charcoal or antioxidants such as vitamin E.

The SofZia® preservative system in Travatan Z (Alcon Laboratories, Fort Worth, Tex.) contains borate, sorbitol, propylene glycol, and zinc. Without intending to be bound by theory, it is believed that the preservative effect is from a combination of borate and zinc. For preservatives including borate and zinc, such as SofZia®, the particulate plug of the disclosure can include a metal chelating agent such as EDTA, anionic hydrogels that can extract cationic zinc through electrostatic interactions, cationic hydrogels or resins that can extract anionic borate ions through electrostatic interactions, or a neutralizing agent that can neutralize boric acid.

In some embodiments, solutions, emulsions, or suspensions of the disclosure comprise a compound or salt of any preservative of the present disclosure, wherein the compound or salt of the preservative is largely free of impurities, such as at least about 80% pure, at least about 81% pure, at least about 82% pure, at least about 83% pure, at least about 84% pure, at least about 85% pure, at least about 86% pure, at least about 87% pure, at least about 88% pure, at least about 89% pure, at least about 90% pure, at least about 91% pure, at least about 92% pure, at least about 93% pure, at least about 94% pure, at least about 95% pure, at least about 96% pure, at least about 97% pure, at least about 98% pure, at least about 99% pure, at least about 99.1% pure, at least about 99.2% pure, at least about 99.3% pure, at least about 99.4% pure, at least about 99.5% pure, at least about 99.6% pure, at least about 99.7% pure, at least about 99.8% pure, or at least about 99.9% pure.

In some embodiments, solutions, emulsions, or suspensions of the disclosure comprise a compound or salt of any preservative of the present disclosure, wherein the preservative is about 70% to about 99.99%, about 80% to about 99.9%, about 85% to about 99%, about 90% to about 99%, about 95% to about 99%, about 97% to about 99%, about 98% to about 99%, about 98% to about 99.9%, about 99% to about 99.99%, about 99.5% to about 99.99%, about 99.6% to about 99.99%, about 99.8 to about 99.99%, or about 99.9% to about 99.99% free of impurities.

The amount of the compound or salt of the preservative in a solution, emulation, or suspension of the present disclosure can be measured as a percentage of mass per volume. In some embodiments, a solution, emulsion, or suspension such as an aqueous solution of the disclosure, comprises from about 0.05 wt % to about 10 wt % of the compound or salt of any of the preservatives disclosed herein. In some embodiments, a solution, emulsion, or suspension such as an aqueous solution of the disclosure, comprises about 0.01 wt %, about 0.02 wt %, about 0.03 wt %, about 0.04 wt %, about 0.05 wt %, about 0.06 wt %, about 0.07 wt %, about 0.08 wt %, about 0.09 wt %, about 0.1 wt %, about 0.2 wt %, about 0.3 wt %, about 0.4 wt %, about 0.5 wt %, about 0.6 wt %, about 0.7 wt %, about 0.8 wt %, about 0.9 wt %, about 1 wt %, about 1.1 wt %, about 1.2 wt %, about 1.3 wt %, about 1.4 wt %, about 1.5 wt %, about 1.6 wt %, about 1.7 wt %, about 1.8 wt %, about 1.9 wt %, about 2 wt %, about 2.1 wt %, about 2.2 wt %, about 2.3 wt %, about 2.4 wt %, about 2.5 wt %, about 2.6 wt %, about 2.7 wt %, about 2.8 wt %, about 2.9 wt %, about 3 wt %, about 3.1 wt %, about 3.2 wt %, about 3.3 wt %, about 3.4 wt %, about 3.5 wt %, about 3.6 wt %, about 3.7 wt %, about 3.8 wt %, about 3.9 wt %, about 4 wt %, about 4.1 wt %, about 4.2 wt %, about 4.3 wt %, about 4.4 wt %, about 4.5 wt %, about 5 wt %, about 6 wt %, about 7 wt %, about 8 wt %, about 9 wt %, or about 10 wt % of a compound or salt of the preservative described herein.

A compound or salt of the preservative described herein can be present in a solution, emulsion, or suspension of the present disclosure at a concentration of, for example, about 500 nM, about 600 nM, about 700 nM, about 800 nM, about 900 nM, about 1 μM, about 2 μM, about 3 μM, about 4 μM, about 5 μM, about 6 μM, about 7 μM, about 8 μM, about 9 μM, about 10 μM, about 20 μM, about 30 μM, about 40 μM, about 50 μM, about 60 μM, about 70 μM, about 80 μM, about 90 μM, about 100 μM, about 150 μM, about 200 μM, about 250 μM, about 300 μM, about 350 μM, about 400 μM, about 450 μM, about 500 μM, about 550 μM, about 600 μM, about 650 μM, about 700 μM, about 750 μM, about 800 μM, about 850 μM, about 900 μM, about 1 mM, about 5 mM, about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, or about 100 mM. The compound of a preservative described herein may be present in a solution, emulsion, or suspension within a range of concentrations, the range being defined by an upper and lower value selected from any of the preceding concentrations. For example, the compound or salt of an preservative of the disclosure may be present in the solution, emulsion, or suspension at a concentration of from about 1 nM to about 100 mM, about 10 nM to about 10 mM, about 100 nM to about 1 mM, about 500 nM to about 1 mM, about 1 mM to about 50 mM, about 10 mM to about 40 mM, about 20 mM to about 35 mM, or about 20 mM to about 30 mM.

### Complexing Agent

In some embodiments, solutions, emulsions, or suspensions of the present disclosure further comprise a complexing agent. In some embodiments, the compound or salt of an ophthalmic agent of the disclosure exhibits high affinity for the matrix material and the addition of a complexing agent reduces the affinity of the ophthalmic agent for the matrix material. In some embodiments, the solution, emulsion, or suspension comprises a cyclodextrin, a linoleic acid, a lipid mixture, an oleic acid, a cholesterol, an arachidonic acid, a cod liver oil, fatty acid, etc. In some embodiments, the solution, emulsion, or suspension is an aqueous solution comprising a complexing agent. In some embodiments, a solution, emulsion, or suspension for topical administration to the eye comprises a complexing agent.

In some embodiments, the ophthalmic agent is hydrophobic. In some embodiments, a polymer matrix material designed to absorb a preservative such as Benzalkonium chloride (BAK) may also absorb a hydrophobic ophthalmic agent. A complexing agent may decrease the affinity of the ophthalmic agent for the matrix material. The matrix material may selectively remove a preservative from the solution, emulsion, or suspension. A complexing agent may be used to tune the interaction between the ophthalmic agent and the matrix. Utilizing a complexing agent, such as cyclodextrin, may change the relative hydrophobicity (hydrophilicity) of the ophthalmic agent relative to the polymer matrix material, thereby decreasing the affinity of the ophthalmic agent for the matrix. Utilizing a complexing agent may keep the ophthalmic agent soluble in the water phase such that it may not be absorbed on or in the polymer matrix material.

As a secondary effect, the capping agent (also called the complexing agent) may increase the solubility of the ophthalmic agent. Due to the relatively low concentrations of ophthalmic agents used herein, solubility may typically not be a concern even if a complexing agent is not used. As an additional secondary effect, the capping agent may increase the stability of a solution comprising the ophthalmic agent and the preservative. As an additional secondary effect, the capping agent may improve the delivery of the ophthalmic agent to certain areas of the body.

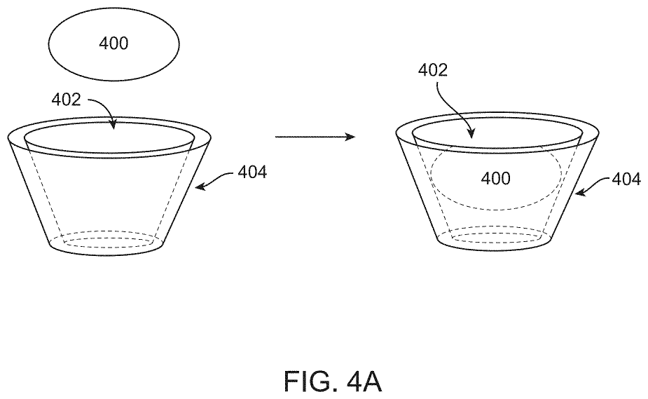

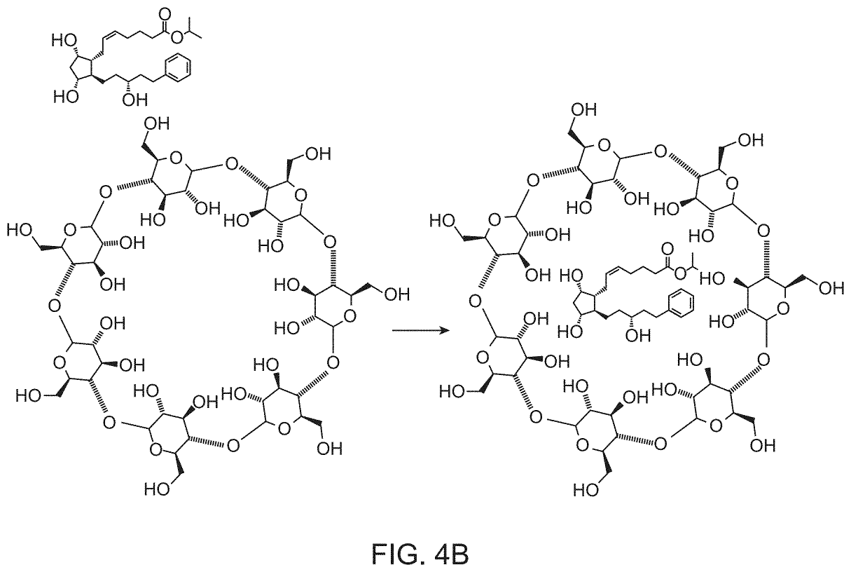

FIG. 4A illustrates a guest-host interaction of a complexing agent and an ophthalmic agent of the present disclosure, in accordance with some embodiments. In some embodiments, the complexing agent (or capping agent) forms a guest-host complex with the ophthalmic agent 400. The complexing agent may have a hydrophobic interior 402 and a hydrophilic exterior 404. In some embodiments, the complexing agent is a cyclodextrin. In some embodiments, the complexing agent is a crown ether. In some embodiments, the complexing agent is a zeolite.

In some embodiments, the complexing agent is a cyclodextrin. A cyclodextrin may comprise glucopyranose sub units. A cyclodextrin may comprise 6, 7, 8, or more glucopyranose units. A cyclodextrin which comprises 6 glucopyranose units may be an alpha cyclodextrin. A cyclodextrin which comprises 7 glucopyranose units may be a beta cyclodextrin. A cyclodextrin which comprises 8 glucopyranose units may be a gamma cyclodextrin. A cyclodextrin may be toroidal in shape with the C2- and C3-hydroxyls forming the larger opening and the C6-hydroxyls forming the smaller opening. The interior of the torus may be hydrophobic. The size of the hydrophobic cavity within the cyclodextrin may be a function of the number of glucopyranose units.

Typical cyclodextrins are constituted by 6-8 glucopyranoside units. These subunits are linked by 1,4 glycosidic bonds. The cyclodextrins have toroidal shapes, with the larger and the smaller openings of the toroid exposing to the solvent secondary and primary hydroxyl groups respectively. Because of this arrangement, the interior of the toroids is not highly hydrophobic, but considerably less hydrophilic than the aqueous environment and thus able to host other hydrophobic molecules. In contrast, the exterior is sufficiently hydrophilic to impart cyclodextrins (or their complexes) water solubility. In some embodiments, the cyclodextrin may be modified by chemical substitution of the hydroxyl groups of the glucopyranose units. Each glucopyranose unit has 3 hydroxyl groups that are available to be reacted and substituted with. In some embodiments multiple of these hydroxyls can be reacted, which is described as degree of substitution. The degree of substitution (DS) describes the number of hydroxyls (on average) that have been reacted. Hydropropoxidation is an example of this type of substitution reaction to create so called hydroxypropyl cycolodextrins of various DS depending how many of the hydroxy groups are reacted. In some embodiments, the cyclodextrin may be (2-hydroxypropyl)-β-cyclodextrin. The cyclodextrin may be (2-hydroxypropyl)-α-cyclodextrin, (2-hydroxypropyl)-γ-cyclodextrin, α-cyclodextrin, β-cyclodextrin, γ-cyclodextrin, methyl-α-cyclodextrin, methyl-β-cyclodextrin, methyl-γ-cyclodextrin, or another substituted cyclic glucose polymer. In other embodiments, the cyclodextrin is chosen from dimethyl-beta-cyclodextrin, highly sulphated-beta-cyclodextrin, 6-monodeoxy-6-N-mono(3-hydroxy)propylamino-beta-cyclodextrin. In other embodiments, the cyclodextrin is a randomly or selectively substituted at the hydroxyls with any chemistry and to any required degree for alpha, beta or gamma or any ring size cyclodextrin. In other embodiments other types of and degrees of substitution on the cyclodextrin rings are also known and possible. Any of these can used as complexing agents. In some embodiments commercial products are possible such as CAVASOL® W7 HP PHARMA is pharmaceutical grade hydroxypropyl-beta-cyclodextrin from Wacker Chemie AG. CAVASOL® W7 HP PHARMA is a highly soluble beta-cyclodextrin derivative. Hydroxypropyl Betadex is another example of this same commercial type cyclodextrin.

In some embodiments, the solution, emulsion, or suspension may comprise the cyclodextrin at a 5000% molar excess over the ophthalmic agent (e.g. a 50 to 1 ratio of cyclodextrin to the ophthalmic agent). The solution, emulsion, or suspension may comprise the cyclodextrin at a greater concentration than the ophthalmic agent. The solution, emulsion, or suspension may comprise the cyclodextrin at a molar excess of greater than 100%, greater than 500%, greater than 1000%, greater than 2000%, greater than 5000%, greater than 10000 or more. The concentration of cyclodextrin may be greater than the ophthalmic agent by a factor of more than 10, by a factor of more than 20, or more.

The molar ratio of a complexing agent of the present disclosure to an ophthalmic agent in a solution, emulsion, or suspension of the present disclosure can be about 200:about 1, about 175:about 1, about 150:about 1, about 125:about 1, about 100:about 1, about 75:about 1, about 65:about 1, about 60:about 1, about 55 about 1, about 50:about 1, about 45:about 1, about 40:about 1, about 30 about 1, about 25:about 1, about 10:about 1, about 9.5:about 1, about 9.0:about 1, about 8.5:about 1, about 8.0:about 1, about 7.5:about 1, about 7.0:about 1, about 6.5:about 1, about 6.0:about 1, about 5.5:about 1, about 5.0:about 1, about 4.5:about 1, about 4.0:about 1, about 3.5:about 1 about 3.0:about 1, about 2.5:about 1, about 2.0:about 1, about 1.9:about 1, about 1.8:about 1, about 1.7:about 1, about 1.6:about 1, about 1.5:about 1, about 1.4:about 1, about 1.3:about 1, about 1.2:about 1, about 1.19:about 1, about 1.18:about 1, about 1.17:about 1, about 1.16:about 1, about 1.15:about 1, about 1.14:about 1, about 1.13:about 1, about 1.12:about 1, about 1.11:about 1. The ratio of a complexing agent to an ophthalmic agent in a solution, emulsion, or suspension of the present disclosure can be within the range of between about 100:about 1 and about 10 to about 1, between about 80:about 1 and about 10:about 1, between about 100:about 1 and about 20:about 1.

In some embodiments, the solution, emulsion, or suspension may comprise the cyclodextrin at a concentration of 127 μM (micromolar). In some embodiments, the solution, emulsion, or suspension may comprise the cyclodextrin at a concentration of greater than 1 μM, 2 μM, 5 μM, 10 μM, 20 μM, 50 μM, 100 μM, or more. In some embodiments, the solution, emulsion, or suspension may comprise the cyclodextrin at a concentration of less than 500 or it may be at a concentration of about 1 mM (millimolar), 2 mM, 5 mM, 10 mM, 20 mM, 50 mM, 100 mM, or less.

In some embodiments, the complexing agent may comprise a mixture of cyclodextrins comprising one or more cyclodextrins disclosed elsewhere herein.

FIG. 4B illustrates a guest-host interaction of a cyclodextrin and Latanoprost, in accordance with some embodiments

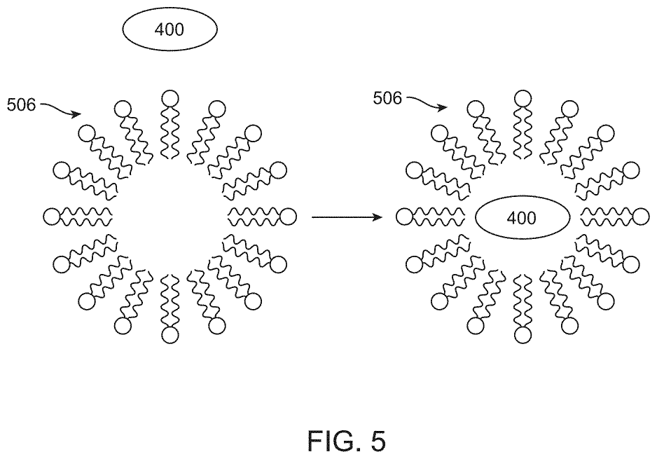

FIG. 5 illustrates a micelle and an ophthalmic agent 400 of the present disclosure, in accordance with some embodiments. In some embodiments, the complexing agent may comprise a micelle forming compound 506. In some embodiments, the complexing agent may comprise a surfactant. The complexing agent may generally comprise an amphiphilic compound. The micelle forming compound may comprise a hydrophilic head group and a hydrophobic tail. The hydrophilic head group may form an exterior surface of the micelle with the hydrophobic tail forming an interior surface of the micelle. The hydrophobic drug may be located inside of the micelle.

The complexing agent may comprise one or more of a linoleic acid, a lipid mixture, an oleic acid, cholesterol, an arachidonic acid, cod liver oil, a fatty acid, etc. In some embodiments a fatty acid may include caprylic acid, capric acid, lauric acid, myristic acid, palmitic acid, stearic acid, arachidic acid, behenic acid, lignoceric acid, or cerotic acid Myristoleic acid, Palmitoleic acid, Sapienic acid, Oleic acid, Elaidic acid, Vaccenic acid, Linoleic acid, Linoelaidic acid, α-Linolenic acid, Arachidonic acid, Eicosapentaenoic acid, Erucic acid, Docosahexaenoic acid or the like.

In some embodiments, a preservative of the present disclosure may be a surfactant. For example, preservatives comprising quaternary ammonium compounds may be surfactants. Purite may be a surfactant. Cetrimide may be a surfactant. In some embodiments, benzalkonium chloride may be a cationic surfactant. Benzalkonium chloride may form micelles. The addition of benzalkonium chloride may stabilize and/or increase the solubility of hydrophobic ophthalmic agents in solution, e.g. latanoprost, bimatoprost, travoprost, etc. Accordingly, hydrophobic ophthalmic agents may be sufficiently solubilized and/or stabilized in formulation comprising benzalkonium chloride. Formulations of hydrophobic ophthalmic agents comprising cyclodextrin may comprise ratios of about 1:1 (agent to cyclodextrin) or may not comprise cyclodextrin at all, as a hydrophobic ophthalmic agent may be sufficiently solubilized without cyclodextrin. For example, marketed ophthalmic formulations of latanoprost may not comprise cyclodextrin as a solubilizing agent.

Without being limited by theory, removal of benzalkonium chloride by the preservative removing device may reduce solubility of a hydrophobic ophthalmic agent in a formulation. In such cases, an amount of a hydrophobic agent, e.g. latanoprost, bimatoprost, travoprost, etc., which may pass through the preservative removing device may be reduced, which may reduce a concentration of the ophthalmic agent in a dose. The addition of a cyclodextrin of the present disclosure may decrease interaction between the hydrophobic agent and a matrix material of the present disclosure. The addition of a cyclodextrin of the present disclosure may maintain solubility of the hydrophobic agent in the formulation as it passes through a matrix material of the present disclosure.

In some embodiments, solutions, emulsions, or suspensions of the disclosure comprise a compound or salt of any complexing agent of the present disclosure, wherein the compound or salt of the complexing agent is largely free of impurities, such as at least about 80 wt % pure, at least about 81% pure, at least about 82% pure, at least about 83% pure, at least about 84% pure, at least about 85% pure, at least about 86% pure, at least about 87% pure, at least about 88% pure, at least about 89% pure, at least about 90% pure, at least about 91% pure, at least about 92% pure, at least about 93% pure, at least about 94% pure, at least about 95% pure, at least about 96% pure, at least about 97% pure, at least about 98% pure, at least about 99% pure, at least about 99.1% pure, at least about 99.2% pure, at least about 99.3% pure, at least about 99.4% pure, at least about 99.5% pure, at least about 99.6% pure, at least about 99.7% pure, at least about 99.8% pure, or at least about 99.9% pure.

In some embodiments, solutions, emulsions, or suspensions of the disclosure comprise a compound or salt of any complexing agent of the present disclosure, wherein the complexing agent is about 70% to about 99.99%, about 80% to about 99.9%, about 85% to about 99%, about 90% to about 99%, about 95% to about 99%, about 97% to about 99%, about 98% to about 99%, about 98% to about 99.9%, about 99% to about 99.99%, about 99.5% to about 99.99%, about 99.6% to about 99.99%, about 99.8 to about 99.99%, or about 99.9% to about 99.99% free of impurities.

The amount of the compound or salt of the complexing agent in a solution, emulation, or suspension of the present disclosure can be measured as a percentage of mass per volume. In some embodiments, a solution, emulsion, or suspension such as an aqueous solution of the disclosure, comprises from about 0.05 wt % to about 10 wt % of the compound or salt of any of the complexing agents disclosed herein. In some embodiments, a solution, emulsion, or suspension such as an aqueous solution of the disclosure, comprises about 0.01 wt %, about 0.02 wt %, about 0.03 wt %, about 0.04 wt %, about 0.05 wt %, about 0.06 wt %, about 0.07 wt %, about 0.08 wt %, about 0.09 wt %, about 0.1 wt %, about 0.2 wt %, about 0.3 wt %, about 0.4 wt %, about 0.5 wt %, about 0.6 wt %, about 0.7 wt %, about 0.8 wt %, about 0.9 wt %, about 1 wt %, about 1.1 wt %, about 1.2 wt %, about 1.3 wt %, about 1.4 wt %, about 1.5 wt %, about 1.6 wt %, about 1.7 wt %, about 1.8 wt %, about 1.9 wt %, about 2 wt %, about 2.1 wt %, about 2.2 wt %, about 2.3 wt %, about 2.4 wt %, about 2.5 wt %, about 2.6 wt %, about 2.7 wt %, about 2.8 wt %, about 2.9 wt %, about 3 wt %, about 3.1 wt %, about 3.2 wt %, about 3.3 wt %, about 3.4 wt %, about 3.5 wt %, about 3.6 wt %, about 3.7 wt %, about 3.8 wt %, about 3.9 wt %, about 4 wt %, about 4.1 wt %, about 4.2 wt %, about 4.3 wt %, about 4.4 wt %, about 4.5 wt %, about 5 wt %, about 6 wt %, about 7 wt %, about 8 wt %, about 9 wt %, or about 10 wt % of a compound or salt of the complexing agent described herein.

A compound or salt of the complexing agent described herein can be present in a solution, emulsion, or suspension of the present disclosure at a concentration of, for example, about 500 nM, about 600 nM, about 700 nM, about 800 nM, about 900 nM, about 1 μM, about 2 μM, about 3 μM, about 4 μM, about 5 μM, about 6 μM, about 7 μM, about 8 μM, about 9 μM, about 10 μM about 20 μM, about 30 μM, about 40 μM, about 50 μM, about 60 μM, about 70 μM, about 80 μM, about 90 μM, about 100 μM, about 150 μM, about 200 μM, about 250 μM, about 300 μM, about 350 μM, about 400 μM, about 450 μM, about 500 μM, about 550 μM, about 600 μM, about 650 μM, about 700 μM, about 750 μM, about 800 μM, about 850 μM, about 900 μM, about 1 mM, about 5 mM, about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, or about 100 mM. The compound of a complexing agent described herein may be present in a solution, emulsion, or suspension within a range of concentrations, the range being defined by an upper and lower value selected from any of the preceding concentrations. For example, the compound or salt of a complexing agent of the disclosure may be present in the solution, emulsion, or suspension at a concentration of from about 1 nM to about 100 mM, about 10 nM to about 10 mM, about 100 nM to about 1 mM, about 500 nM to about 1 mM, about 1 mM to about 50 mM, about 10 mM to about 40 mM, about 20 mM to about 35 mM, or about 20 mM to about 30 mM.

### Excipients

Devices and methods of the present disclosure may comprise formulating the solution, emulsion, or suspension with one or more inert, pharmaceutically-acceptable excipients. Liquid compositions include, for example, solutions in which a compound is dissolved, emulsions comprising a compound, or a solution containing liposomes or micelles comprising an ophthalmic agent as disclosed herein. These compositions can also contain minor amounts of nontoxic, auxiliary substances, such as wetting or emulsifying agents, pH buffering agents, tonicity agents and other pharmaceutically-acceptable additives.

In some embodiments, solutions, emulsions, or suspensions of the present disclosure further comprise one or more physiologically acceptable carriers including excipients and auxiliaries which facilitate processing of the pharmaceutical agent into preparations which are used pharmaceutically. Proper formulation is dependent upon the route of administration chosen.

Pharmaceutically acceptable carriers include, for example, aqueous solutions such as water or physiologically buffered saline or other solvents or vehicles such as glycols, glycerol, oils such as olive oil, or organic esters. The excipients can be chosen, for example, to effect delayed release of an agent or to selectively target one or more cells, tissues or organs. The composition can also be present in a solution suitable for topical administration, such as an eye drop.

Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, hydroxypropyl methylcellulose, hypromellose, Methocel, methyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

In some embodiments, the solutions, emulsions, or suspensions of the disclosure may include one or more additional excipients. The amount of the excipient in a pharmaceutical formulation of the disclosure can be about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1%, about 1.5%, about 2%, about 2.5%, about 3%, about 3.5%, about 4%, about 4.5%, about 5%, about 6%, about 7%, about 8%, about 9%, about 10%, about 15%, about 20%, about 25%, about 30%, about 35%, about 40%, about 45%, about 50%, about 60%, about 70%, about 80%, about 90%, about 100%, about 200%, about 300%, about 400%, about 500%, about 600%, about 700%, about 800%, about 900%, or about 1000% by mass of the compound in the solution, emulsion, or suspension. The amount of the excipient in a solution, emulsion, or suspension of the disclosure can be between 0.01% and 1000%, between 0.02% and 500%, between 0.1% and 100%, between 1% and 50%, between 0.01% and 1%, between 1% and 10%, between 10% and 100%, between 50% and 150%, between 100% and 500%, or between 500% and 1000% by mass of the compound in the solution, emulsion, or suspension.

The amount of the excipient in a solution, emulsion, or suspension of the present disclosure can be about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1%, about 1.5%, about 2%, about 2.5%, about 3%, about 3.5%, about 4%, about 4.5%, about 5%, about 6%, about 7%, about 8%, about 9%, about 10%, about 15%, about 20%, about 25%, about 30%, about 35%, about 40%, about 45%, about 50%, about 55% about 60%, about 65%, about 70%, about 75%, about 80%, about 85%, about 90%, about 95%, about 99%, or about 100% by mass or by volume of the unit dosage form. The amount of the excipient in a solution, emulsion, or suspension can be between 0.01% and 1000%, between 0.02% and 500%, between 0.1% and 100%, between 1% and 50%, between 0.01% and 1%, between 1% and 10%, between 10% and 100%, between 50% and 150%, between 100% and 500%, or between 500% and 1000% by mass or by volume of the unit dosage form.

The ratio of a compound of an ophthalmic agent of the present disclosure to an excipient in a pharmaceutical formulation of the present disclosure can be about 100:about 1, about 95:about 1, about 90:about 1, about 85:about 1, about 80:about 1, about 75:about 1, about 70:about 1, about 65:about 1, about 60:about 1, about 55:about 1, about 50:about 1, about 45:about 1, about 40:about 1, about 35:about 1 about 30:about 1, about 25:about 1, about 20:about 1, about 15:about 1, about 10:about 1, about 9:about 1, about 8:about 1, about 7:about 1, about 6:about 1, about 5:about 1, about 4:about 1, about 3:about 1, about 2:about 1, about 1:about 1, about 1:about 2, about 1:about 3, about 1:about 4, about 1:about 5, about 1:about 6, about 1:about 7, about 1:about 8, about 1:about 9, or about 1:about 10. The ratio of a compound of an ophthalmic agent to an excipient in a solution, emulsion, or suspension of the present disclosure can be within the range of between about 100:about 1 and about 1 to about 10, between about 10:about 1 and about 1:about 1, between about 5:about 1 and about 2:about 1.

In some embodiments, a solution, emulsion, or suspension of the present disclosure comprises an agent for adjusting the pH of the formulation. In some embodiments, the agent for adjusting the pH could be an acid, e.g., hydrochloric acid or boric acid, or a base, e.g., sodium hydroxide or potassium hydroxide. In some embodiments, the agent for adjusting the pH is an acid such as boric acid. The formulation may comprise about 0.05 wt % to about 5 wt %, about 0.1% to about 4%, about 0.1% to about 3 wt %, about 0.1 wt % to about 2 wt %, or about 0.1 wt % to about 1 wt % of an agent for adjusting the pH.

Solutions, emulsions, or suspensions of the disclosure can be formulated at any suitable pH. In some embodiments, the pH of the solution emulsion or suspension is about 4, about 4.05, about 4.1, about 4.15, about 4.2, about 4.25, about 4.3, about 4.35, about 4.4, about 4.45, about 4.5, about 4.55, about 4.6, about 4.65, about 4.7, about 4.75, about 4.8, about 4.85, about 4.9, about 4.95, about 5, about 5.1, about 5.2, about 5.3, about 5.4, about 5.5, about 5.6, about 5.7, about 5.8, about 5.9, about 6, about 6.1, about 6.2, about 6.3, about 6.4, about 6.5, about 6.6, about 6.7, about 6.8, about 6.9, about 7, about 7.1, about 7.2, about 7.3, about 7.4, about 7.5, about 7.6, about 7.7, about 7.8, about 7.9, about 8, about 8.1, about 8.2, about 8.3, about 8.4, about 8.5, about 8.6, about 8.7, about 8.8, about 8.9, or about 9 pH units. In some embodiments, the pH of the solution, emulsion, or suspension is from about 4 to about 10, about 4.75 to about 7.40, about 5 to about 9, about 6 to about 8, about 6.5 to about 8, about 7 to about 8, about 7.2 to about 8, about 7.2 to about 7.8, about 7.3 to about 7.5, or about 7.35 to about 7.45. In some embodiments the pH of the solution, emulsion, or suspension is about 7.4.

In some embodiments, the addition of an excipient to a pharmaceutical formulation of the present disclosure can increase or decrease the viscosity of the composition by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%. In some embodiments, the addition of an excipient to a pharmaceutical formulation of the present disclosure can increase or decrease the viscosity of the composition by no greater than 5%, no greater than 10%, no greater than 15%, no greater than 20%, no greater than 25%, no greater than 30%, no greater than 35%, no greater than 40%, no greater than 45%, no greater than 50%, no greater than 55%, no greater than 60%, no greater than 65%, no greater than 70%, no greater than 75%, no greater than 80%, no greater than 85%, no greater than 90%, no greater than 95%, or no greater than 99%. Examples of ranges which the viscosity change falls within can be created by combining any two of the preceding percentages. For example the addition of an excipient can increase or decrease the viscosity of the composition by 5% to 99%, by 10% to 95%, by 20% to 70% or by 35% to 55%.

In some embodiments, an excipient that increases a viscosity may comprise polyvinyl alcohol, poloxamers, hyaluronic acid, carbomers, and polysaccharides, that is, cellulose derivatives, hydroxymethyl cellulose, hypromellose, Methacel, gellan gum, and xanthan gum. In some embodiments, an excipient that increases mucoadhesive properties may be added. Excipients that increase mucoadhesion may include polyacrylic acid, hyaluronic acid, sodium carboxymethyl cellulose, lectins, and chitosan.

In some embodiments, solutions, emulsions, or suspensions of the present disclosure further comprise an agent for adjusting the osmolarity of the solution, emulsion, or suspension, e.g., mannitol, sodium chloride, sodium sulfate, dextrose, potassium chloride, glycerin, propylene glycol, calcium chloride, and magnesium chloride. In some embodiments, the solution, emulsion, or suspension comprises from about 0.1 wt % to about 10 wt %, about 0.5 wt % to about 8 wt %, about 1 wt % to about 5 wt %, about 1 wt % to about 4 wt %, or about 1 wt % to about 3 wt % of an agent for adjusting the osmolarity of the solution, emulsion, or suspension. In some embodiments, the solution, emulsion, or suspension of the disclosure has an osmolarity from about 10 mOsm to about 1000 mOsm, about 100 mOsm to about 700 mOsm, about 200 mOsm to about 400 mOsm, about 250 mOsm to about 350 mOsm or about 290 mOsm to about 310 mOsm.

In some embodiments, solutions, emulsions, or suspensions of the present disclosure further comprise a buffering agent, such as tromethamine, potassium phosphate, sodium phosphate, saline sodium citrate buffer (SSC), acetate, saline, physiological saline, phosphate buffer saline (PBS), 4-2-hydroxyethyl-1-piperazineethanesulfonic acid buffer (HEPES), 3-(N-morpholino)propanesulfonic acid buffer (MOPS), and piperazine-N,N′-bis(2-ethanesulfonic acid) buffer (PIPES), sodium acetate-boric acid stock solution, boric acid-sodium carbonate with sodium chloride solution, boric acid-sodium borate buffer, sodium and potassium phosphate buffers, boric acid-sodium carbonate with potassium chloride, or combinations thereof. In some embodiments, the solution, emulsion, or suspension comprises from about 0.05 wt % to about 5 wt %, about 0.1 wt % to about 4 wt %, about 0.1 wt % to about 3 wt %, about 0.1 wt % to about 2 wt %, or about 0.1 wt % to about 1 wt % of an agent for buffering the solution, emulsion, or suspension.

In some embodiments, the solution emulsion or suspension provided herein comprises an alcohol as an excipient. Non-limiting examples of alcohols include ethanol, propylene glycol, glycerol, polyethylene glycol, chlorobutanol, isopropanol, xylitol, sorbitol, maltitol, erythritol, threitol, arabitol, ribitol, mannitol, galactilol, fucitol, lactitol, and combinations thereof.

### Salts

Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

The compounds may be synthesized using conventional techniques. Advantageously, these compounds are conveniently synthesized from readily available starting materials. Synthetic chemistry transformations and methodologies useful in synthesizing the compounds described herein are known in the art.

The present disclosure provides salts of any one or both of an ophthalmic agent and a preservative. Pharmaceutically-acceptable salts include, for example, acid-addition salts and base-addition salts. The acid that is added to the compound to form an acid-addition salt can be an organic acid or an inorganic acid. A base that is added to the compound to form a base-addition salt can be an organic base or an inorganic base. In some embodiments, a pharmaceutically-acceptable salt is a metal salt.

Metal salts can arise from the addition of an inorganic base to a compound of the present disclosure. The inorganic base consists of a metal cation paired with a basic counterion, such as, for example, hydroxide, carbonate, bicarbonate, or phosphate. The metal can be an alkali metal, alkaline earth metal, transition metal, or main group metal. In some embodiments, the metal is lithium, sodium, potassium, cesium, cerium, magnesium, manganese, iron, calcium, strontium, cobalt, titanium, aluminum, copper, cadmium, or zinc.

In some embodiments, a metal salt is an ammonium salt, a lithium salt, a sodium salt, a potassium salt, a cesium salt, a cerium salt, a magnesium salt, a manganese salt, an iron salt, a calcium salt, a strontium salt, a cobalt salt, a titanium salt, an aluminum salt, a copper salt, a cadmium salt, or a zinc salt.

Ammonium salts can arise from the addition of ammonia or an organic amine to a compound of the present disclosure. In some embodiments, the organic amine is triethyl amine, diisopropyl amine, ethanol amine, diethanol amine, triethanol amine, morpholine, N-methylmorpholine, piperidine, N-methylpiperidine, N-ethylpiperidine, dibenzylamine, piperazine, pyridine, pyrazole, pipyrazole, imidazole, pyrazine, or pipyrazine.

In some embodiments, an ammonium salt is a triethyl amine salt, a diisopropyl amine salt, an ethanol amine salt, a diethanol amine salt, a triethanol amine salt, a morpholine salt, an N-methylmorpholine salt, a piperidine salt, an N-methylpiperidine salt, an N-ethylpiperidine salt, a dibenzylamine salt, a piperazine salt, a pyridine salt, a pyrazole salt, an imidazole salt, or a pyrazine salt.

Acid addition salts can arise from the addition of an acid to a compound of the present disclosure. In some embodiments, the acid is organic. In some embodiments, the acid is inorganic. In some embodiments, the acid is hydrochloric acid, hydrobromic acid, hydroiodic acid, nitric acid, nitrous acid, sulfuric acid, sulfurous acid, a phosphoric acid, isonicotinic acid, lactic acid, salicylic acid, tartaric acid, ascorbic acid, gentisinic acid, gluconic acid, glucuronic acid, saccharic acid, formic acid, benzoic acid, glutamic acid, pantothenic acid, acetic acid, propionic acid, butyric acid, fumaric acid, succinic acid, methanesulfonic acid, ethanesulfonic acid, benzenesulfonic acid, p-toluenesulfonic acid, citric acid, oxalic acid, or maleic acid.

In some embodiments, the salt is a hydrochloride salt, a hydrobromide salt, a hydroiodide salt, a nitrate salt, a nitrite salt, a sulfate salt, a sulfite salt, a phosphate salt, isonicotinate salt, a lactate salt, a salicylate salt, a tartrate salt, an ascorbate salt, a gentisinate salt, a gluconate salt, a glucuronate salt, a saccharate salt, a formate salt, a benzoate salt, a glutamate salt, a pantothenate salt, an acetate salt, a propionate salt, a butyrate salt, a fumarate salt, a succinate salt, a methanesulfonate (mesylate) salt, an ethanesulfonate salt, a benzenesulfonate salt, a p-toluenesulfonate salt, a citrate salt, an oxalate salt, or a maleate salt.

The methods and formulations described herein include the use of amorphous forms as well as crystalline forms (also known as polymorphs). Active metabolites of compounds or salts of any one of the compounds of the present disclosure having the same type of activity are included in the scope of the present disclosure. In addition, the compounds described herein can exist in unsolvated as well as solvated forms with pharmaceutically acceptable solvents such as water, ethanol, and the like. The solvated forms of the compounds and salts presented herein are also considered to be disclosed herein.

In some embodiments, an aqueous solutions, emulsions, or suspensions of the disclosure comprises at least 90 wt % water, such as at least 91 wt %, at least 92 wt %, at least 93 wt %, at least 94 wt %, at least 95 wt %, at least 96 wt %, at least 97 wt %, at least 98 wt %, or even at least 99 wt % of water.

### Preservative Removal Agent

The present disclosure provides a preservative removal agent (e.g. a matrix). A preservative removal agent may rapidly and selectively remove preservatives of the present disclosure from a solution, emulsion, or suspension comprising an ophthalmic agent. The preservative removal agent may rapidly and selectively extract the preservative, allowing the eye drop formulation to flow through the plug with minimal pressure drop, yet with sufficient time to remove the preservative and with sufficient surface area and chemistry to adsorb the preservative. The matrix may comprise a material with a high affinity for the preservative, such as for example benzalkonium chloride (BAK), and at the same time a low affinity for a drug or other ophthalmological agent especially in this invention when the drug is also in the complex with a complexing of capping agent. The preservative removal agent may be sufficiently selective, such that at least 50 percent of the preservative may be removed and at least 50 percent of the drug may be retained by the solution. BAK (benzalkonium chloride) can also go under a number of synonyms: alkylbenzyldimethylammonium chloride, alkyldimethylbenzylammonium chloride, benzyl ammonium chloride to name a few. It is also defined by a structure such as Formula: C₆H₅CH₂N(CH₃)₂RCl (R=C₈H₁₇ to C₁₈H₃₇) with a CAS Number: 63449-41-2. For most purposes in ophthalmic applications and formulations PharmaGrade, EP, USP, JP, manufactured under appropriate GMP controls for pharma or biopharmaceutical production is used.

Non-limiting examples of a preservative removal agents may comprise solid, gel, and/or particulate matrices. The preservative removal agent may act as a physical barrier or filter. Additionally, or alternatively, the preservative removal agent may chemically remove a preservative such as by adsorption of the preservative onto the matrix. The preservative removal agent may be disposed in the outlet of a container, which container may contain the solution, emulsion, or suspension.

In some embodiments, a matrix disposed within a nozzle may be a porous polymeric matrix. The porous polymeric matrix may comprise a variety of materials. Such material may be safe and biocompatible. Such material may comprise but is not limited to, for example, Poly(2-hydroxyethyl methacrylate) (pHEMA), poly(hydroxylethyl methacrylate-co-methacrylic acid), crosslinked polyacrylamide, dimethyl acrylamide, methyl methacrylate, silicones, and/or any combination of the preceding materials.

In some embodiments, the matrix may be highly porous. The pore size in the matrix may be small enough so that the molecules, which may initially be far from the surface of the polymer in the matrix, may diffuse towards the polymer and adsorb. A matrix may have large interconnected pores which may allow flow of solution and adsorption of the preservative into the pores. The matrix may be formed as a porous gel, as a packed bed, and/or a structure formed by 3D printing soft lithography, electrospinning, or any other appropriate method. In some embodiments, the matrix may comprise a microporous gel. In some embodiments, the matrix may comprise a packed bed of pHEMA or crosslinked polyacrylamide or other polymeric particles. The particles may be macroporous. The particles may be spherical or non-spherical. In some embodiments, the polymeric matrix may comprise nano or micron sized polymeric particles (e.g., nanogels or microgels). In some embodiments, the polymeric matrix may comprise a cryogel. In some embodiments, the polymeric matrix may be termed a hydrogel, be hydrophilic and absorb water readily. In some embodiments, the particles themselves may directly impart the preservative effect, such as colloidal silver nanoparticles.

In certain embodiments, particles of the formulations described herein have an average diameter from about 1 nm to about 10 μm, about 1 nm to about 10 μm, about 1 nm to about 5 μm, about 1 nm to about 2 μm, about 1 nm to about 1 μm, about 1 nm to about 900 nm, about 1 nm to about 800 nm, about 1 nm to about 700, about 1 nm to about 600 nm, about 1 nm to about 500 nm, about 1 nm to about 400 nm, about 1 nm to about 300 nm, about 1 nm to about 200 nm, or even from about 1 nm to about 100 nm. In certain embodiments, the average diameter is the average largest diameter or the average equivalent diameter.

In certain embodiments, greater than 80% of the particles, such as greater than 90% or greater than 95% of the particles in the formulation have an average largest particle diameter of from about 1 nm to about 1000 μm, about 1 nm to about 10 μm, about 1 nm to about 5 μm, about 1 nm to about 2 μm, about 1 nm to about 1 μm, about 1 nm to about 900 nm, about 1 nm to about 800 nm, about 1 nm to about 700, about 1 nm to about 600 nm, about 1 nm to about 500 nm, about 1 nm to about 400 nm, about 1 nm to about 300 nm, about 1 nm to about 200 nm, or even from about 1 nm to about 100 nm. In certain embodiments, the average diameter is the average largest diameter or the average equivalent diameter.

In certain embodiments, particles of the porous polymeric matrix described herein have an average diameter from about 100 nm to about 10 μm, about 100 nm to about 10 μm, about 100 nm to about 5 μm, about 100 nm to about 2 μm, about 100 nm to about 1 μm, about 100 nm to about 900 nm, about 100 nm to about 800 nm, about 100 nm to about 700, about 100 nm to about 600 nm, about 200 nm to about 500 nm, about 250 nm to about 600 nm, about 300 nm to about 600 nm, about 350 nm to about 700 nm, about 450 nm to about 550 nm, about 475 nm to about 525 nm, or from about 400 nm to about 700 nm. In certain embodiments, the average diameter is the average largest diameter or the average equivalent diameter.

In certain embodiments, greater than 80% of the particles of the porous polymeric matrix, greater than 90% of the particles of the porous polymeric matrix, or greater than 95% of the particles of the porous polymeric matrix have an average diameter from about 100 nm to about 10 μm, about 100 nm to about 10 μm, about 100 nm to about 5 μm, about 100 nm to about 2 μm, about 100 nm to about 1 μm, about 100 nm to about 900 nm, about 100 nm to about 800 nm, about 100 nm to about 700, about 100 nm to about 600 nm, about 200 nm to about 500 nm, about 250 nm to about 600 nm, about 300 nm to about 600 nm, about 350 nm to about 700 nm, about 450 nm to about 550 nm, about 475 nm to about 525 nm, or from about 400 nm to about 700 nm. In certain embodiments, the average diameter is the average largest diameter or the average equivalent diameter.

In certain embodiments, greater than 80% of the particles of the porous polymeric matrix, greater than 90% of the particles of the porous polymeric matrix, or greater than 95% of the particles in the formulation have an average diameter from about 10 μm to about 100 μm, about 50 μm to about 200 μm, about 90 μm to about 180 μm, about 150 μm to about 250 μm, about 200 μm to about 350 μm about 250 μm to about 500 μm, about 350 μm to about 800 μm, about 500 μm to about 1000 μm In certain embodiments, the average diameter is the average largest diameter or the average equivalent diameter. The particles may be irregular, regular, spherical, ovoid, or generally of any shape and the size can be defined as passing through a certain sized screen sieve.

The matrix may comprise a tortuosity such that the flow path of a solution, emulsion, or suspension through the nozzle may be significantly increased. In an embodiment where the matrix is a packed bed of macroporous particles, the packed beds of macroporous particles may have three levels of porosity: the space between the particles, the macropores in the particles, and the inherent porosity of the polymer. In such an embodiment, all three levels of porosity may contribute to the tortuosity of the matrix.

In some embodiments, a matrix disposed within a nozzle may be a porous polymeric matrix. Applying a pressure behind the nozzle may cause fluid to flow through the nozzle via the flow path, along which path the preservative may be removed by adsorption onto the matrix. The polymer material, the hydraulic permeability, the partition coefficient, the adsorption rate, and the pore size in combination may aid in the absorption of all or most of the preservative from the solution and thus patient eye drops. The reduced preservative solution may subsequently be delivered directly to the eye. The porous polymeric matrix may rapidly and selectively extract the preservative, allowing the eye drop formulation to flow through the plug with minimal pressure drop, yet with sufficient time to remove the preservative and with sufficient surface area to adsorb the preservative. The matrix may comprise a material with a high affinity for the preservative, such as for example benzalkonium chloride (BAK), and low affinity for a drug or other ophthalmological agent. The porous polymeric matrix may comprise a high affinity for the preservative, such that at least 50 percent of the preservative may be removed and at least 50 percent of the drug may be retained by the solution.

The porous polymeric matrix may comprise a variety of materials. Such material are safe and biocompatible. A polymer of the present disclosure may comprise various monomers, for example, Poly(2-hydroxyethyl methacrylate) (pHEMA) and/or and/or acrylamide (AM), dimethyl acrylamide (DMA) and/or methyl methacrylate (MMA) and/or N-Vinylpyrrolidone (NVP) and/or 2-acrylamido-2-methylpropane sulfonic acid (AMPS) and/or polyvinyl alcohol (PVA) and/or polymethylpropane sulfonic acid (PAMPS) and/or 2-sulfoethyl methacrylate (SEM) and/or acrylic acid (AA) and/or vinylphosphonic acid (VP) and/or t-butyl methacrylate (TBM) and/or Methacryloxypropyltris(trimethylsiloxy)silane (TRIS) and/or t-amyl methacrylate and/or n-octyl methacrylate and/or iso-decyl methacrylate and/or n-decyl methacrylate and/or n-dodecyl acrylate and/or n-hexyl acrylate and/or n-dodecyl acrylate and/or N-(n-Octadecyl)acrylamide and/or silicones and/or any combination of the preceding materials. The polymeric matrix may further comprise a cross linker. A crosslinker may comprise N,N′-methylenebis(acrylamide) (MBAM) and/or triacrylamido triazine (TATZ) and/or SR 351 and/or SR9035 and/or any combination of the preceding materials.

In some embodiments, the matrix material is a copolymer. A copolymer may comprise more than one species of monomer. Copolymers may be branched. Copolymers may be linear. Copolymers may comprise crosslinkers. Copolymers may be block copolymers, may be alternating copolymers, may be periodic copolymers, may be gradient copolymers may be statistical copolymers, may be stereoblock copolymers. The copolymers may exhibit phases of differing hydrophobicity or hydrophilicity. The hydrophobicity and/or hydrophilicity of the one or more monomers or cross-linkers may control the binding of a therapeutic agent or a preservative to the plug material.

In some embodiments, the polymeric matrix is polyvinyl alcohol crosslinked with citric acid or other suitable crosslinking agent to render it a hydrophilic hydrogel. In some embodiments, the polymeric matrix is crosslinked polyvinylpyrrolidone, crosslinked polyethylene oxide, crosslinked polyacrylamides, crosslinked copolymers of methacrylic acid, polyacrylic acid and copolymers such as poly (acrylic acid-co-acrylamide), or poly (methacrylic acid-co-acrylamide).

Polymers of the present disclosure may generally follow an A/B/C formula where A and B are monomers, C is one or more cross-linkers, and A and B are not the same monomer. In some examples, A may be an anionic hydrophilic monomer. In an A/B/C formula, monomers of type A may comprise AM or NVP. In some examples, B may be an ionic hydrophilic monomer. In an A/B/C formula, monomers of type B may comprise MAA, AMPS, SEM, AA, or VP. In some examples, C may be a crosslinker. In an A/B/C formula, monomers of type C may comprise one or more of MBAM, TATZ, or SR 351. Polymers of the present disclosure may generally follow an A/C formula where A is a monomer as described above and C is one or more cross-linkers as described above. Polymers of the present disclosure may generally follow an B/C formula where B is a monomer as described above and C is one or more cross-linkers as described above.

Polymers of the present disclosure may also comprise grafted copolymers such that components such as monomer A and with a cross-linker C are first copolymerized to form a crosslinked copolymer that can be isolated as a small bead or other shaped particle. These beads or particles can then be reswollen in water and a monomer of B type can added and then polymerized into or onto the bead or particle through the use a free radical “grafting” polymerization. In this embodiment the particles are made up of A/C copolymer with a “grafted” B polymer as part of the copolymer structure.

The following is a non-exhaustive list of examples of polymers of the present disclosure. The following includes polymer components and percent compositions, separated by slashes, respectively, and an identifier corresponding to an example polymer in Example 3 and Example 4. Polymers of the present disclosure may comprise: AMPS/MBAM/TATZ 7.5/82.5/10 (D-322-018-AW), AMPS/MBAM/TATZ 7.5/77.5/15 (D-322-020-AW), AMPS/MBAM 7.5/92.5 (D-322-022-AW), BioRad Beads/AMPS 1 g/0.5 (D-322-028-C-AW), AMPS/MBAM 7.5/92.5 (D-322-002-AG-W), AMPS/MBAM/TATZ 7.5/87.5/5.0 (D-322-006-AW), SEM/MBAM 7.5/92.5 (D-322-010-AW), AM/2-Sulfoethyl MA(SEM)/MBAM 30/10/60 (D-298-132-A), AMPS/MBAM 7.5/92.5 (D-298-190-AW); AMPS/MBAM 7.5/92.5 (D-298-196-A), AMPS/MBAM 7.5/92.5 (D-298-196-AW), AMPS/MBAM 7.5/92.5 (D-298-178-AW), PVA/PAMPS/CA 4.8/1.2/2.4 IPN (D-298-182-A), AMPS/MBAM 7.5/92.5 ISP (D-298-184-AW), NVP/AMPS/MBAM/TATZ 30/10/30/30 (D-298-186-A), AMPS/MBAM 7.5/92.5 (D-298-152-AW), N-vinylpyrrolidinone/AMPS/MBAM 30/10/60 (D-298-120-AW), AA/SR351 40/60 (D-298-146-A), AA/MBAM/SR351 60/30/10 (D-298-148-A), AM/2-Sulfoethyl MA (SEM)/MBAM 15/25/60 (D-298-134-A), AA/MBAM 40/60 (D-298-140-A), AA/MBAM 50/50 (D-298-142-A), and VP/AA/MBAM 10/45/45 (D-298-144-A).

Any matrix material and any drug in association with a complexing agent may be used such that the drug/complex partition coefficient into the matrix may be lower by at least an order of magnitude or 2 orders of magnitude than the matrix's affinity for the preservative. For example, pHEMA, or SO3- or PO3H— or COO— groups on the polymer (or matrix) may bind BAK with a partition coefficient of about 100-500, or in some embodiments, 1000 depending on the BAK concentration and the structure of the matrix and the % content of those groups. In some embodiments, the matrix may comprise a partition coefficient for the preservative from the solution, emulsion, or suspension of, for example, at least 10, at least 100, at least 1000, at least 10,000, or within a range defined by any two of the preceding values. Additionally, or alternatively, the adsorption rate constant may be sufficiently high so that the time for adsorption of a drug molecule to the polymer may be less than the time to form a drop. The time to form a drop may comprise a time within a range from 0.1 to 10 seconds.

The matrix may display a high hydraulic permeability such that relatively little pressure may be required to dispense a fluid. The hydraulic permeability may depend on the design of the filter. Larger pores in the matrix may allow for higher flow for a given pressure drop. In some embodiments, hydraulic permeability may be larger than about 0.01 Darcy. A nozzle may comprise a permeability of about 0.1 Darcy. A hydraulic permeability of 1 to 10 Darcy may allow fluid to be retained in the filter during instances when the pressure may be lowered subsequent to formation of a drop. A larger hydraulic permeability may allow the same plug to work for a wide range of formulations including, for example, high viscosity formulations, such as rewetting eye drops. In some embodiments, the porous polymeric matrix comprises a hydraulic permeability of, for example, 0.01 Da, 0.1 Da, 1 Da, 10 Da, 100 Da, 1000 Da or a hydraulic permeability within a range defined by any two of the preceding values.

In some embodiments, the matrix may be highly porous. The pore size in the matrix may be small enough so that the molecules, which may initially be far from the surface of the polymer in the matrix, may diffuse towards the polymer and adsorb. A matrix may comprise large interconnected pores which may allow flow of solution and adsorption of the preservative into the pores. The matrix may be formed as a porous gel, as a packed bed, and/or a structure formed by 3D printing soft lithography, electrospinning of a fiber, or any other appropriate method. In some embodiments, the matrix may comprise a microporous gel. In some embodiments, the matrix may comprise a packed bed of pHEMA or crosslinked polyacrylamide with an anionic moiety or functionality as part of the polymer or other polymeric particles. The particles may be macroporous. The particles may be spherical or non-spherical. In some embodiments, the polymeric matrix may comprise nano or micron sized or 10s of microns or 100s of microns of polymeric particles (e.g., nanogels or microgels). In some embodiments, the polymeric matrix may comprise a cryogel. In some embodiments, the particles themselves may directly impart the preservative effect, such as colloidal silver nanoparticles.

In some embodiments, the particles may need to be stably held in the nozzle and prevented from eluting from the nozzle. The particles may be attached to the container walls through long polymeric chains and/or by placing a filter at the exit from the device. Additionally, or alternatively, the walls of the container or other surfaces may comprise preservative attached thereupon and/or incorporated therein. In such embodiments, the preservative source comprises a pHEMA membrane with 1-10% by volume equilibrated with BAK. In some embodiments, the matrix comprises pre-loaded with BAK at a concentration to inhibit microbial growth over time.

In some embodiments, the porous matrix material may comprise a tortuosity such that the flow path of a solution, emulsion, or suspension through the nozzle increases. In some embodiments where the matrix comprises a packed bed of macroporous particles, the packed beds of macroporous particles may comprise three levels of porosity: the space between the particles, the macropores in the particles, and the inherent porosity of the polymer. In such embodiments, all three levels of porosity may contribute to the tortuosity of the matrix. The tortuosity of the porous material combined with the geometry nozzle itself may increase the flow path in accordance with a multiplicative factor of a first flow path length corresponding to flow defined by the nozzle geometry and a second flow path length corresponding to the tortuosity of the porous material.

The pressure needed for drop creation may exceed the Young Laplace pressure during drop creation, which may be about 2σ/R_{d} where σ is the surface tension and R_{d} is the radius of the drop. Estimating R_{d}˜0.5 mm based on a drop volume of 30 μL, and using the surface tension of water may yield a Young Laplace pressure of about 100 Pa. The pressure to form a drop may additionally exceed the pressure needed to displace 30 μL of volume. Typical drop volumes may comprise a volume within a range between 1 μL and 100 μL. The minimum pressure to form a drop may be ˜0.01 Atm (1000 Pa) based on an ideal gas estimate using a 3 mL bottle at atmospheric pressure, but may be lower for larger bottles at varying pressures. Maximum pressure to form a drop may be limited by a patient strength. The pressure to form a drop may be within a range between 0.01 Atm and 0.5 Atm.

The rate of liquid flow through the plug may depend on the applied pressure as well as the design parameters of the matrix including, but not limited to, length, area, porosity, hydraulic permeability, flow path length, etc. These design parameters may be considered individually or in combination to remove preservative without excessive squeeze pressure. The rate of liquid flow may affect the time to form a drop.

### System: Definitions of Solution a and Solution B are Found in Example

A drop of solution A that has been passed through the porous polymeric hydrogel B has a concentration of Latanoprost of at least 80% of the original concentration of Latanoprost in solution A. Said drop has more preferably 90% of the original concentration of Latanoprost in solution A. And most preferably >95% of the original concentration of Latanoprost in solution A.

In addition, a drop of solution A that has been passed through the porous polymeric hydrogel B has a concentration of total BAK of less than 50% of the original concentration of the BAK in the original concentration of BAK in solution A. Said drop has more preferably less than 20% and more preferably still less than 5% of the original concentration of BAK in solution A. And most preferably <1% or below detection limits by someone skilled in the art of the original concentration of BAK in solution A.

In addition, a drop of solution A that has been passed through the porous polymeric hydrogel B has a concentration of BAK of less than 10% of the original concentration of the BAK in the original concentration of BAK in solution A. Said drop has more preferably less than 5% of the original concentration of BAK in solution A. And most preferably <1% or non-detectable by standard methods such as HPLC of the original concentration of BAK in solution A.

### Dosage

The dosage and frequency (single or multiple doses) administered to a mammal may vary depending upon a variety of factors, for example, whether the mammal suffers from another disease, and its route of administration; size, age, sex, health, body weight, body mass index, and diet of the recipient; nature and extent of symptoms of the disease being treated, kind of concurrent treatment, complications from the disease being treated or other health-related problems. Other therapeutic regimens or agents may be used in conjunction with the methods and compounds of this disclosure. Adjustment and manipulation of established dosages (e.g., frequency and duration) are well within the ability of those skilled in the art.

Dosages may be varied depending upon the requirements of the patient and the compound being employed. The dose administered to a patient, in the context of the present disclosure should be sufficient to affect a beneficial therapeutic response in the patient over time. The size of the dose also may be determined by the existence, nature, and extent of any adverse side effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals may be adjusted individually to provide levels of the administered compound effective for the particular clinical indication being treated. This may provide a therapeutic regimen that is commensurate with the severity of the individual's disease state

### EXAMPLES

It is understood that the examples and embodiments described herein are for illustrative purposes only and are not intended to limit the scope of the claimed invention. It is also understood that various modifications or changes in light of the examples and embodiments described herein will be suggested to persons skilled in the art and are to be included within the spirit and purview of this application and scope of the appended claims. All publications, patents, and patent application cited herein are hereby incorporated by reference in their entirety for all purposes.

It will be understood that various ophthalmic agents may be used in any aspect of the disclosure provided. It will be understood that various cyclodextrins may be used in any aspect of the disclosure provided to complex the ophthalmic agent in aqueous solution. It will be understood that various preservatives may be used in any aspect of the disclosure provided to render the original solution stable for storage. Porous polymeric hydrogel A as prepared and used in the examples described herein is done so for demonstration purposes. It will be understood that various porous polymer hydrogel materials may be used in any aspect of the disclosure provided.

### Example 1

### Solution a was Prepared in the Following Manner.

50:1 molar ratio of 2-(hydroxypropyl)-β-cyclodextrin: latanoprost solution was prepared by first adding 1.6768 gm (1.1565×10⁻² moles) of 2-(hydroxypropyl)-β-cyclodextrin (Hydroxypropyl Betadex is a partially substituted poly(hydroxypropyl) ether of Betadex). The number of hydroxypropyl groups per anhydroglucose unit expressed as molar substitution (MS) is not less than 0.40 and not more than 1.50 and is within 10 percent of the value stated on the label.) to 2000 ml of distilled water at 25° C. in a vessel with high agitation under nitrogen atmosphere until all the cyclodextrin was dissolved. With continued agitation, 0.1 gm (2.313×10⁻⁴ moles) of latanoprost was added and mixing was continued at 25° C. until a clear solution was observed to ensure complete dissolution.

0.4 gm of Benzalkonium Chloride (BAK) CAS Number: 63449-41-2, available from Aldrich Chemical, product number 12063, PharmaGrade, EP, USP, JP, manufactured under appropriate GMP controls for pharma or biopharmaceutical production was added to the solution and mixing was continued at 25° C. to ensure a homogenous clear solution.

The concentration of latanoprost in this solution was 0.005% and BAK was 0.02% (by weight). The latanoprost is complexed with the 2-(hydroxypropyl)-β-cyclodextrin. There was a 50:1 1 mole ratio of the cyclodextrin to the Latanoprost.

### Porous Polymer Hydrogel B was Prepared in the Following Manner:

The materials in the table below were used in the procedure for hydrogel B:

| Cmpd | mol ratio | wt ratio | amt | | comments |
|---|---|---|---|---|---|
| SEM | 0.075 | — | 2.62 | g | 180 total mmol |
| | | | | | monomers |
| MBAM | 0.925 | — | 25.67 | g | |
| water | | | 410 | mL | 14 volumes |
| KPS | 0.02 | — | 0.973 | g | initiator |

2-Sulfoethyl methacrylate (SEM) obtained from Polysciences catalog number 02597-50G×2

N,N′-Methylenebisacrylamide (MBAM) obtained from Sigma-Aldrich cat. No. 146072-100G

Potassium persulfate (KPS) obtained from Sigma-Aldrich cat. no. 21622-100G Purified distilled and deionized water.

Porous hydrogel polymer was prepared as follows. A 500 mL reactor with single turbine blade mechanical stirrer was heated in water bath. A solution of SEM (2.62 g) and MBAM (25.67 g) in 400 mL of water was prepared in the reactors, and the mixture was heated to 55° C. KPS (0.973 g in 10 mL of water) was added via syringe. The temperature was increased to 60° C. for 6 hours. The product was worked-up by centrifuge concentration of the gel material formed (copolymer) followed by washing with IPA and water in 3 times each with centrifuging to concentrate between each wash. The solid was collected by filtration on Whatman #1 paper and dried in a vacuum oven. The resulting solid powder was place in a soxhlet and extracted with IPA. It was further extracted with water in the soxhlet. The purified solid was removed from the soxhlet filter, dried under vacuum and sieved to obtain a powder particle fraction 250-500 microns in size.

The procedure for demonstrating the selective absorption of the BAK preservative from solution A by passing through the porous polymeric hydrogel B (both prepared as described herein) was described previously in U.S. Pat. No. 10,123,904, which is incorporated herein by reference in its entirety. Another procedure (analytical method) is the use of quantitative HPLC using a partition coefficient procedure or a simple equilibrium test to compare the area under the curve (AUC) of the starting solution for drug and BAK vs the AUC for the solution in contact with the hydrogel at room temperature equilibrium. In that case, a skilled analyst can calculate the percent of both the drug and the BAK at equilibrium remaining in the contact solute. In the present invention it is desirable to have a very high percentage (>90%) of the drug unabsorbed by the hydrogel copolymer while also having a high percentage (>50%) of the BAKs (usually BAK C12 and BAK C14) absorbed by the hydrogel copolymer at equilibrium for example after 48 hrs at room temperature. An example of a partition coefficient (PC) test was performed as follows. The test hydrogel copolymer (0.1 g) was weighed into a small vial. To that was added 5.00 ml of the latanoprost with cyclodextrin complex formulation with BAK (such as described in Solution A). The vial was sealed and then gently swirled to contact the liquid with the solid test hydrogel. The vial was allowed to sit at room temperature for 48 hours. Then, the liquid was separated from the solids through a syringe with a filter and analyzed via HPLC to measure the amount of latanoprost and BAK at equilibrium. The area under the curves for latanoprost and for the BAKs in the starting solution were then compared to the AUC for the solute separated from the hydrogel after equilibrium. In this way, a percentage of the drug and a percentage of the BAKs was measured after contact with the hydrogel.

### Example 2

Comparative Solution B (without CD) was Prepared in the Following Manner.

0.1 gm (2.313×10⁻⁴ moles) of Latanoprost was mixed with 2000 ml of distilled water at 25° C. in a vessel with high agitation under nitrogen atmosphere for several hours to ensure complete dissolution. 0.4 gm of Benzalkonium Chloride (BAK) was added to the solution and mixing was continued at 25° C. to ensure a homogenous clear solution. The concentration of latanoprost in solution B was 0.005% and BAK was 0.02% (by weight)

The procedure for demonstrating the selective absorption of the BAK preservative from solution B by passing through the porous polymeric hydrogel B (both prepared as described herein) was described previously in U.S. Pat. No. 10,123,904, which is incorporated by reference herein in its entirety. Another procedure (analytical method) is the use of quantitative HPLC using a partition coefficient procedure or a simple equilibrium test to compare the area under the curve (AUC) of the starting solution for drug and BAK vs the AUC for the solution in contact with the hydrogel at room temperature equilibrium. In that case, a skilled analyst can calculate the percent of both the drug and the BAK at equilibrium remaining in the contact solute. In the present invention it is desirable to have a very high percentage (>90%) of the drug unabsorbed by the hydrogel copolymer while also having a high percentage (>50%) of the BAKs (usually BAK C12 and BAK C14) absorbed by the hydrogel copolymer at equilibrium for example after 48 hrs at room temperature.

The results of the Example 1 and the comparative Example 2 are shown in Table 1. The results show that the effective latanoprost concentration in solution after passing through the porous polymer hydrogel was greater than 90% of the original concentration whilst the BAK concentration was reduced to 34% of its original concentration. The comparative example 2 that has no cyclodextrin to complex the latanoprost shows that both the latanoprost and the BAK were absorbed in large measure by passing the solution through the hydrogel. In this case there is not enough effective, therapeutic, ophthalmic agent available in solution after passing through the porous polymer hydrogel. These results demonstrate that a formulation of the present disclosure may benefit from the use of the complexing agent (such as a cyclodextrin) in the solution with the ophthalmic agent. A complexing agent may keep the agent in solution after contact with a hydrogel that has the structure and chemistry to absorb the preservative (such as BAK) from said solution.

TABLE 1
Summary of Results: Example 1 vs Example 2

| | | Example 1. | | Comparative |
|---|---|---|---|---|
| | | Solution A, | | Example 2 |
| | Example 1. | CD/Latanoprost | Comparative | Solution B, |
| | Solution A, | Complex plus | Example 2. | Latanoprost |
| | CD/Latanoprost | BAK after | Solution B, | plus BAK after |
| | Complex plus | passing through | Latanoprost | passing through |
| | BAK | hydrogel B | plus BAK | hydrogel B |
| Latanoprost | 5.00 × 10⁻² | 4.725 × 10⁻² | 5.00 × 10⁻² | 1.75 × 10⁻² |
| Concentration | mg/ml | mg/ml | mg/ml | mg/ml |
| | | 94.5% | | 34.9% |
| | | unabsorbed | | unabsorbed |
| Total BAK | 20.00 × 10⁻2 | 6.8 × 10⁻² | 20.00 × 10⁻² | 0.04 × 10⁻² |
| Concentration | mg/ml | mg/ml | mg/ml | mg/ml |
| | | 66.0% | | 99.8% |
| | | absorbed | | absorbed |

### Example 3

### Procedure for Producing Hydrogel Crosslinked Copolymer Hydrogels:

This same basic procedure was used for all hydrogels in Example 3 included in this section. The monomer quantities and monomer materials and crosslinker quantities and crosslinker materials were varied and the initiator materials and initiator quantities were varied as described in the individual hydrogels listed here as Example 3. The procedure for preparing, separating, collecting, purifying, and drying the hydrogels in this example are as follows:

### Components:

a. Acrylamide, or N-Vinylpyrrolidone (NVP), monomer;
b. Methacrylic acid or 2-Acrylamido-2-methylpropane sulfonic acid (AMPS), or 2-Sulfolethyl methacrylate (SEM), or Acrylic Acid, or Vinylphosphonic acid;
c. N,N′-Methylenebis(acrylamide) (MBAM) Aldrich number 146072 or triacrylamido triazine (TATZ), or SR 351, or other crosslinkers.

A free radical initiated polymerization reaction vessel was equipped for mechanical agitation. The vessel was charged with 300 ml of distilled water and degassed with nitrogen bubbling purge through the water for 10 minutes. Fifty grams of total mixture of the 3 monomers (a, b, and c) are charged at the desired ratio with stirring at 300 rpm. Potassium persulfate (2 g) is added to the reactor and heated to 60° C. with 300 agitator speed. The desired copolymer became a gel phase and then began to precipitate as a gel mass. Continue stirring for 3 hours at 60° C. to complete the reaction. The resulting hydrogel was collected by centrifugation, washed with 2× volumes of water then filtered and dried to final powder and ground to fine powder form.

The hydrogel polymer was purified using a soxhlet extractor using a 2× extraction first with isopropyl alcohol (IPA) and then a 2× extraction with pure water. The final polymer was ground and sieved to desired particle size for testing.

### Preparation of Hydrogel

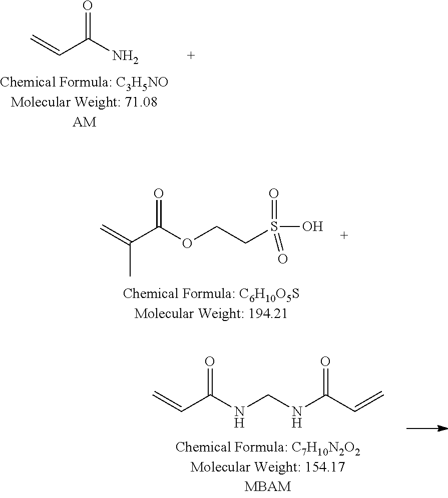

Monomer molar ratios: Acrylamide:2-sulfoethylmethacrylate (SEM):MBAM (crosslinker)/10:30:60.

The gel-like material was compressed via centrifugation (5000 rpm for 15 min). Washed with 2×50 mL of IPA, and then washed with 2×50 mL water. Dried under vacuum at 50-60° C. 35.95 g obtained. Ground up and sieved. D-298-132-A, 500 μm to 250 μm, 6.542 g; D-298-132-B, <250 μm, 28.672 g.

### D-298-134-A and B

Monomer molar ratios: Acrylamide:2-sulfoethylmethacrylate:MBAM (crosslinker)/15:25:60.

The gel-like material was compressed via centrifugation (5000 rpm for 15 min). Washed with 2×50 mL of IPA, and then washed with 2×50 mL water. Dried under vacuum at 50-60° C. 36.70 g obtained. Ground up and sieved. D-298-134-A, 500 μm to 250 μm, 10.924 g; D-298-134-B, <250 μm, 23.750 g.

### D-298-140

Monomer molar ratios: N-vinylpyrrolidinone:Acrylic acid:MBAM (crosslinker)/0:40:60.

The granular material was compressed via centrifugation (5000 rpm for 15 min). Washed with 30% aqueous IPA (2×), then washed with water (2×). Dried under vacuum at 50-60° C. 33.84 g obtained. Ground up and sieved. D-298-140-A, 500 μm to 250 μm, 6.040 g; D-298-140-B, <250 μm, 3.871 g.

### D-298-142

Monomer molar ratios: N-vinylpyrrolidinone:Acrylic acid:MBAM (crosslinker)/0:50:50.

The material was compressed via centrifugation (5000 rpm for 15 min). Washed with 30% aqueous IPA (2×), then washed with water (2×). Dried under vacuum at 50-60° C. The granular material collected upon grinding and sieving was 250-500 microns in size.

### D-298-144

Monomer molar ratios: N-vinylpyrrolidinone:Acrylic acid:MBAM (crosslinker)/10:45:45.

The granular material was compressed via centrifugation (5000 rpm for 15 min). Washed with 30% aqueous IPA (2×), then washed with water (2×)). Dried under vacuum at 50-60° C.

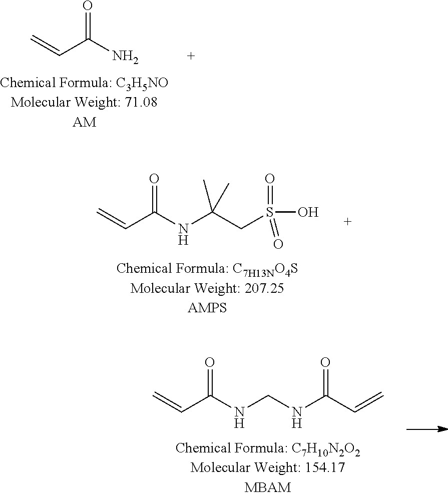

### D-298-152-AW

Monomer molar ratios: Acrylamide (AM):2-Acrylamido-2-methylpropane sulfonic acid AMPS: N,N′-Methylenebis(acrylamide) MBAM (crosslinker)/0:7.5:92.5.

The gel-like material was compressed via centrifugation (5000 rpm for 15 min). Washed with 30% aqueous IPA (2×), then washed with water (2×). Dried under vacuum at 50-60° C.

Washed again with 2×50 mL of IPA, and then washed with 2×50 mL water. Dried under vacuum at 50-60° C. 27.75 g obtained. Ground up and sieved. D-298-152-AW, 500 μm to 250 μm, 6.555 g; D-298-152-B, <250 μm, 21.864 g.

### D-298-178 (Repeat Run of D-298-152)

Monomer molar ratios: AMPS:MBAM (crosslinker); 7.5:92.5.

The gel-like material was compressed via centrifugation (5000 rpm for 15 min). Washed with 2×50 mL of IPA, and then washed with 2×50 mL water. Dried under vacuum at 50-60° C. 28.87 g obtained. Ground up and sieved. D-298-178-AW, 500 μm to 250 μm, 16.730 g, D-298-178-B, <250 μm, 12.332 g.

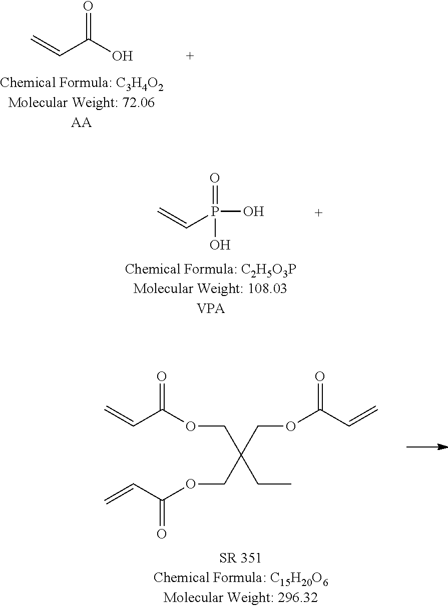

### D-298-164

Monomer molar ratios: Acrylic acid:vinyl phosphonic acid:SR 351 (crosslinker) trifunctional trimethylolpropane triacrylate (TMPTA) grade. SR 351 available from Sartomer (Arkema Group)/65:30:5.

A very small amount of solid was obtained. The gel-like material was compressed via centrifugation (5000 rpm for 15 min). Washed with water. Dried under vacuum at 50-60° C.

### D-298-166

Monomer molar ratios: Acrylicacid:vinylphosphonicacid:SR 351 (crosslinker)/47.5:47.5:5.

A small amount of solid was obtained. The gel-like material was compressed via centrifugation (5000 rpm for 15 min). Washed with water. Dried under vacuum at 50-60° C.

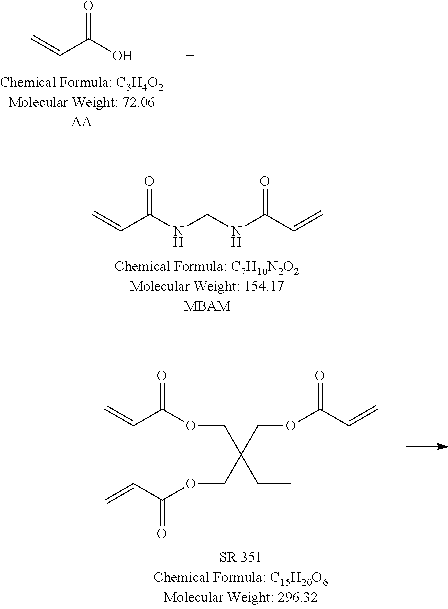

### D-298-146-A

Monomer molar ratios: Acrylic acid:MBAM (crosslinker):SR 351 (crosslinker)/40:0:60.

The granular material was compressed via centrifugation (5000 rpm for 15 min). Washed with 30% aqueous IPA (2×), then washed with water (2×). Dried under vacuum at 50-60° C. 34.80 g obtained. Ground up and sieved. D-298-146-A, 500 μm to 250 μm, 7.722 g; D-298-146-B, <250 μm, 4.166 g.

### D-298-148-A

Monomer molar ratios: Acrylic acid:MBAM (crosslinker):SR 351 (crosslinker)/60:30:10. The granular material was compressed via centrifugation (5000 rpm for 15 min). Washed with 30% aqueous IPA (2×), then washed with water (2×). Dried under vacuum at 50-60° C.

### D-298-190:

The following quantities were used and procedure as described:

| Cmpd | mol ratio | wt ratio | amt | | comments |
|---|---|---|---|---|---|
| AMPS | 0.075 | — | 1.943 | g | |
| MBAM | 0.925 | — | 17.83 | g | |
| PVP40 | — | 0.02 | 0.394 | g | 2% of |
| (emulsifier) | | | | | monomer mass |
| KPS | 0.005 | — | 0.169 | g | initiator |
| water | | | 350 | mL | |
| cyclohexane | | | 300 | mL | |

Solids formed after 10 minutes, and heating continued another 5 hours. After cooling overnight, the product was worked-up by centrifugation as described. The centrifuge cups were cut open, and two were oven-dried under vacuum at 50-60° C., and the other two were freeze-dried.

D-298-190-AW, oven-dried, ground, sieved to 250-500 μM: 2.159 g
D-298-190-FD-A, freeze-dried, 250-500 μM: 0.298 g

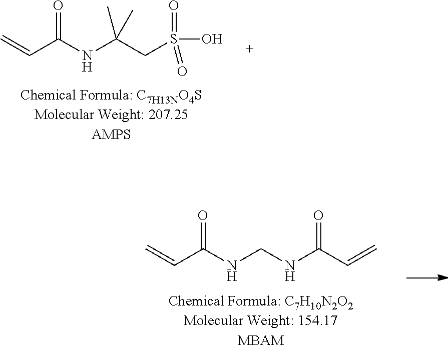

TABLE 2
D-298-196 (additional KPS)

| Cmpd | mol ratio | wt ratio | amt | | comments |
|---|---|---|---|---|---|
| AMPS | 0.075 | — | 2.72 | g | 175 total mmol |
| | | | | | monomers |
| MBAM | 0.925 | — | 24.96 | g | |
| water | | | 400 | mL | 14 volumes |
| KPS-1 | 0.02 | — | 0.946 | g | initiator |
| KPS-2 | 0.01 | — | 0.473 | g | initiator |

After 3 hours of reaction time, an additional charge of KPS was made, and the reaction was heated for another 4 hours. The gel-like material was compressed via centrifugation (5000 rpm for 15 min). Washed with 2×50 mL of IPA, and then washed with 2×80 mL water. Dried under vacuum at 50-60° C. 25.95 g obtained. Ground up and sieved. D-298-196-A, 500 μm to 250 μm, 13.744 g; D-298-196-B, <250 μm, 11.114 g.

A portion of D-298-196-A (1.70 g) was purified by water extraction in a soxhlet. The solid was air-died at 50-60° C. for 2 days and sieved. D-298-196-AW, 500 μm to 250 μm, 0.919 g.

### D-322-002

(repeat of D-298-196, additional KPS, air-dried) The reaction was run on the same scale as D-298-196. After 3 hours of reaction time, an additional charge of KPS was made, and the reaction was heated for another 4 hours. The gel-like material was compressed via centrifugation (5000 rpm for 15 min). Washed with 2×50 mL of IPA, and then washed with 2×80 mL water. Air-dried at 50-60° C. 29.31 g obtained. Dried solid was sieved. D-322-002-A, 500 μm to 250 μm, 3.889 g, D-322-002-B, <250 μm, 3.93 g.

The rest of the material was ground up and sieved. D-322-002-AG-W, 500 μm to 250 μm, 12.342 g, D-322-002-BG, <250 μm, 8.50 g

A portion of D-322-002-AG (3.50 g) was purified by IPA extraction in a soxhlet, followed by water extraction in a Soxhlet, dried and sieved.

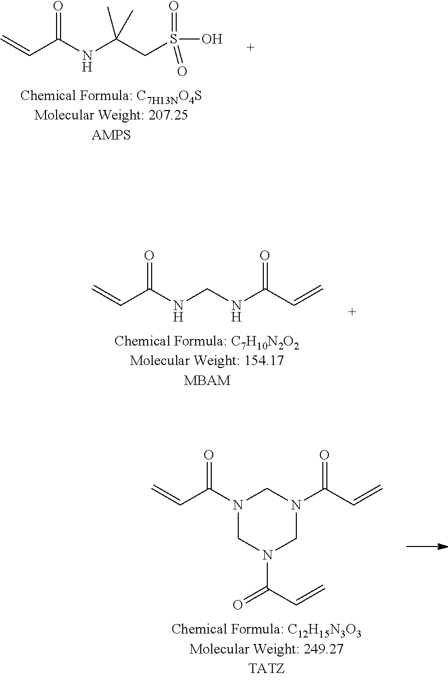

TABLE 3
D-322-006 trifunctional cross-linker with improved particle integrity

| Cmpd | mol ratio | wt ratio | amt | | comments |
|---|---|---|---|---|---|
| AMPS | 0.075 | — | 2.72 | g | 175 total mmol |
| | | | | | monomers |
| MBAM | 0.875 | — | 23.6073 | g | |
| TATZ | 0.05 | | 2.181 | g | |
| water | | | 412 | mL | 14 volumes |
| KPS | 0.02 | — | 0.946 | g | initiator |

The reaction was run as normal. The slurry was compressed via centrifugation (5000 rpm for 15 min). Washed with 2×50 mL of IPA, and then washed with 2×80 mL of water. Dried under vacuum at 50-60° C. 25.26 g obtained. Ground up and sieved. D-322-006-A, 500 μm to 250 μm, 14.728 g; D-322-006-B, <250 μm, 9.344 g.

A portion of D-322-006-A (3.50 g) was purified by IPA extraction in a soxhlet, followed by water extraction in a soxhlet. Then the product hydrogel is dried and sieved as desired.

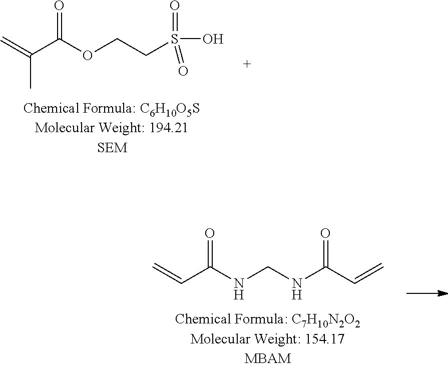

TABLE 4
D-322-010-AW (2-sulfoethylmethacrylate (SEM) copolymer)

| Cmpd | mol ratio | wt ratio | amt | comments |
|---|---|---|---|---|
| SEM | 0.075 | — | 2.62 g | 180 total mmol monomers |
| MBAM | 0.925 | — | 25.67 g | |
| water | | | 410 mL | 14 volumes |
| KPS | 0.02 | — | 0.973 g | initiator |

The reaction was run as normal. The slurry was compressed via centrifugation (5000 rpm for 15 min). Washed with 2×50 mL of IPA, and then washed with 2×80 mL of water. Dried under vacuum at 50-60° C.

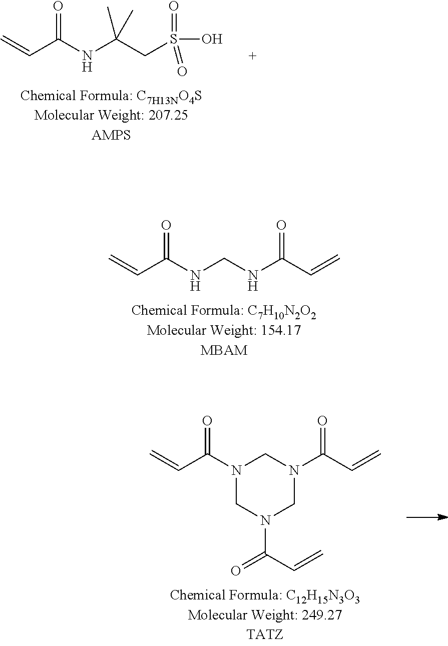

TABLE 5
D-322-018 trifunctional cross-linker TATZ, 10%

| Cmpd | mol ratio | wt ratio | amt | comments |
|---|---|---|---|---|
| AMPS | 0.075 | — | 2.80 g | 180 total mmol monomers |
| MBAM | 0.825 | — | 22.89 g | |
| TATZ | 0.10 | | 4.49 g | |
| water | | | 436 mL | 14 volumes |
| KPS | 0.02 | — | 0.973 g | initiator |

The reaction was run as normal. The slurry was compressed via centrifugation (5000 rpm for 15 min). Washed with 2×50 mL of IPA, and then washed with 2×80 mL of water. Dried under vacuum at 50-60° C.

TABLE 6
D-322-020 trifunctional cross-linker TATZ, 15%

| Cmpd | mol ratio | wt ratio | amt | comments |
|---|---|---|---|---|
| AMPS | 0.075 | — | 2.80 g | 180 total mmol monomers |
| MBAM | 0.775 | — | 21.51 g | |
| TATZ | 0.15 | | 6.73 g | |
| water | | | 448 mL | 14 volumes |
| KPS | 0.02 | — | 0.973 g | initiator |

The reaction was run as normal. The slurry was compressed via centrifugation (5000 rpm for 15 min). Washed with 2×50 mL of IPA, and then washed with 2×80 mL of water. Dried under vacuum at 50-60° C.

### D-298-120 AW

Monomer molar ratios: N-vinylpyrrolidinone:AMPS:MBAM (crosslinker) 30:10:60.

The gel-like material was compressed via centrifugation (5000 rpm for 15 min). Washed with 2×50 mL of IPA, and then washed with 2×50 mL water. The solid was dried under vacuum at 50-60° C.

Experiments with Bio-Rad Beads

Bio Gel P-4 beads were purchased directly from Bio-Rad Corporation of Hercules Calif. Bio-Gel P gels are described as porous polyacrylamide beads prepared by copolymerization of acrylamide (AM) and N,N′-methylene-bis-acrylamide (MBAM). The gels are extremely hydrophilic and essentially free of charge, and provide efficient, gentle gel filtration of sensitive compounds. Their synthetic composition and freedom from soluble impurities preclude eluate contamination. High resolution is assured by consistent narrow distribution of bead diameters and excellent molecular weight discrimination These were used without further purification.

### D-322-028-C

To a slurry of Bio Gel P-4 beads (1.0 g) in 10 mL of water was added AMPS (50 wt %, 500 mg, 2.412 mmol), and the mixture was heated to 45° C. to dissolve the AMPS. KPS (2 mol %, 48.3 mg, 1.206 mL of 40 mg/mL solution in water). The temperature was increased to 60° C. for 6 hours. The product was worked-up by centrifuge washing with IPA and water. The solid was collected by filtration, dried in a vacuum oven. The dried solid was sieved, D-322-028-CA, 0.350 g, 250 μm to 125 μm.

### D-322-028-D, -E Precipitation Polymerization in the Presence of Beads

TABLE 7
Charge Table per 20 mL vial

| Cmpd | D-322-028-D | D-322-028-E |
|---|---|---|
| AMPS | 0.0933 g | 0.0933 g |
| MBAM | 0.856 g | 0.856 g |
| water | 13.3 mL (14 vol) | 13.3 mL (14 vol) |
| Bio-Rad beads | 0.25 g | 0.50 g |
| KPS | 32.4 mg (0.81 mL) | 32.4 mg (0.81 mL) |

To a slurry of the beads in 13.3 mL of water was added MBAM and AMPS. The slurry was heated to 45° C. to dissolve the MBAM and KPS (2 mol %, 32.4 mg, 0.81 mL of 40 mg/mL solution in water) was added. The temperature was increased to 60° C. for 6 hours. The products were worked-up by centrifuge washing with IPA and water. The solids were dried in the tubes in a vacuum oven. The dried solids were ground, sieved and purified by soxhlet extraction with IPA and water. D-322-028-D-AW, 500 μm to 250 μm, 0.4986 g, D-322-028-D-BW, <250 μm, 0.0666 g. D-322-028-E-AW, 500 μm to 250 μm, 0.5058 g, D-322-028-E-BW, <250 μm, 0.1239 g.

### D-322-040 10% SEM/MBAM Hydrogel

TABLE 8

| Cmpd | mol ratio | amt | comments |
|---|---|---|---|
| SEM | 0.10 | 3.88 g | 200 total mmol monomers |
| MBAM | 0.90 | 27.75 g | |
| water | | 475 mL | 15 volumes |
| KPS | 0.02 | 1.08 g | initiator |

The reaction was run as normal. The slurry was compressed via centrifugation (5000 rpm for 15 min). Washed with 2×50 mL of IPA, and then washed with 2×80 mL of water. Dried under vacuum at 50-60° C. 30.79 g obtained. Ground up and sieved. D-322-040-A, 500 μm to 250 μm, 17.403 g, D-322-040-B, ≤250 μm, 12.968 g.

A portion of D-322-040-A (5.0 g) was purified by IPA extraction in a soxhlet, followed by water extraction in a soxhlet. It was dried and re-sieved to give D-322-040-AW, 3.45 g.

### D-322-056 SEM Addition (Grafted) to Bio-Gel P-4 Bio-Rad Beads (BRB P-4)

TABLE 9

| Cmpd | wt ratio | amt | comments |
|---|---|---|---|
| SEM | 0.5 | 15.0 g | 77.24 total mmol monomers |
| BRB P-4 | 1.0 | 30.0 g | Used as received from Bio- |
| | | | Rad Corporation, Hercules, |
| | | | CA |
| water | | 300 mL | 10 volumes |
| KPS | 0.02 | 0.418 g | initiator |

To a solution of SEM in of water was added the beads, and the mixture was heated to 55° C. KPS (solution in water) was added via syringe. The temperature was increased to 60° C. for 6 hours. The product was worked-up by centrifuge washing with IPA and water in 3×250 mL tubes. A portion of the solid was filtered directly into a fritted soxhlet cup (D-322-056-02). D-322-056-02 was soxhlet extracted with IPA, the solid shrank to about half of its volume. It was further extracted with water, whereby it resumed its original volume. The purified solid was filtered, dried under vacuum, and sieved. D-322-056-02-AW, 500 μm to 250 μm, 6.21 g.

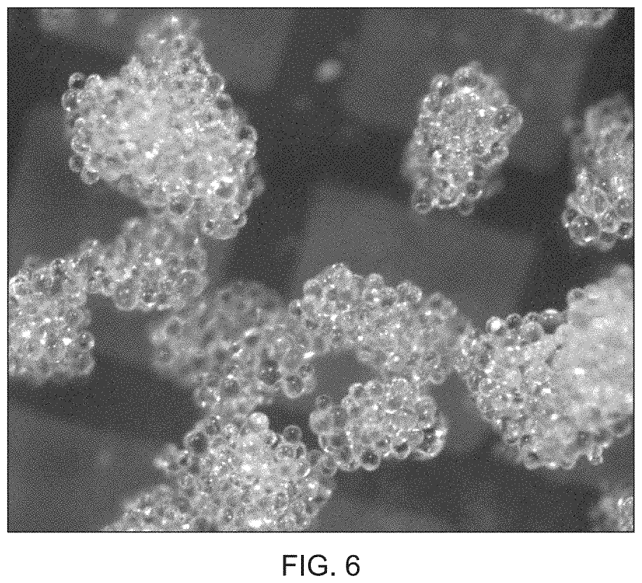

FIG. 6 provides an example optical microscope image of hydrogel D-322-056 described above.

D-298-184-A and AW: Alternate polymerization technique using Inverse Phase Polymerization (ISP) preparation of AMPS/MBAM 7.5/92.5.

The procedure here was followed: In a 500 mL reactor was added MBAM (17.83 g) and AMPS (1.94 g). Water (150 mL) was added, and the mixture was stirred and heated to −40° C. An additional 100 mL of water was needed to dissolve. Xylene (250 mL) containing 0.42 g of ethyl cellulose was added. Heating to −50° C. was continued, as stirring was increased to 310 rpm. A nice emulsion formed. KPS (0.2 g in 10 mL water) was added, and heating stabilized at 60° C. Solids formed after 10 minutes, and heating continued another 4 hours. After cooling overnight, the product was worked-up by centrifugation as described previously. The final isolation was on a Whatman #1 paper filter, 11 cm. The product was dried under vacuum at 50-60° C. to give 14.73 g. The dried solid was sieved gently without mechanical grinding. The cut from 500-250 μm (D-298-184-A), 2.035 g, was purified via soxhlet extraction:
a. I-propyl alcohol (IPA) was used as the extraction solvent in the soxhlet for 4 hours.
b. Water was used as the extraction solvent in the soxhlet for 6 hours.

The washed material was dried under vacuum at 50-60° C., and re-sieved, D-298-184-AW.

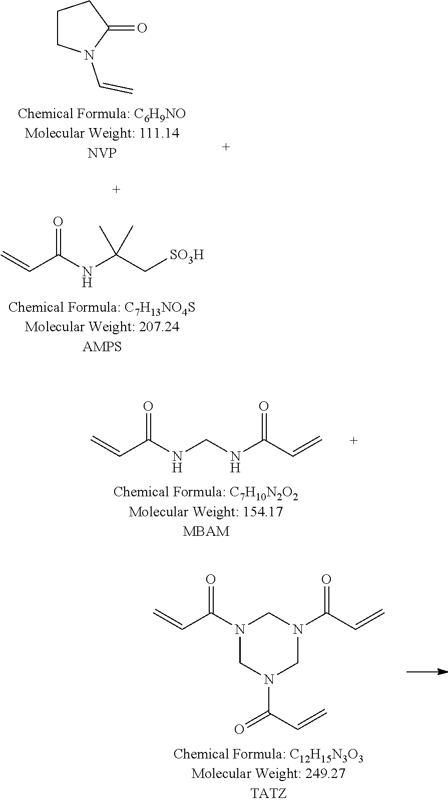

### D-298-186-AW and B

Monomer molar ratios: AMPS:N-vinylpyrrolidinone (NVP):MBAM (crosslinker): TATZ (crosslinker); 10:30:30:30.

The slurry was compressed via centrifugation (5000 rpm for 15 min). Washed with 2×50 mL of IPA, and then washed with 2×50 mL water. The product was collected on a Whatman #1 paper filter, and was dried under vacuum at 50-60° C. 18.53 g obtained. Ground up and sieved. D-298-186-AW, 500 μm to 250 μm, 9.215 g, D-298-186-B, <250 μm, 5.975 g.

### Example 4

Use of Interpenetrating Networks (IPNs) with Modifications as Hydrogels:

These examples show the utility of IPNs in this invention. These can be used as the polymeric absorbing hydrogels as well as the copolymer examples shown in Example 3 or elsewhere in this patent.

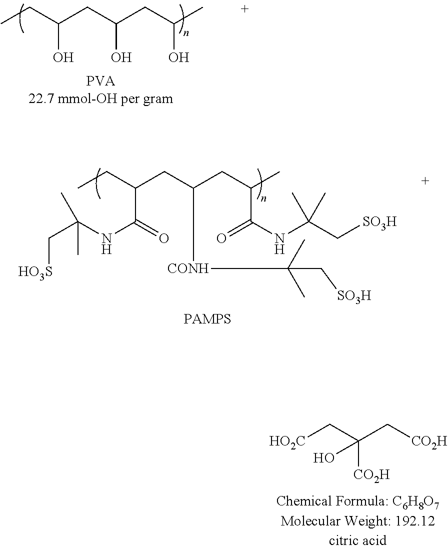

### D-298-182

Monomer weight ratios (g): Polyvinyl alcohol (PVA) (89-98K): Poly AMPS (PAMPS) (15% aq): citric acid; 4.8:1.2:2.4 were used to prepare a citric acid modified IPN of PVA and PAMPS. The 5% total concentration in water was mixed until dissolved and then poured into small aluminum pans and allowed to dry overnight in a vented hood. Much of the water dried off leaving a rubber like film of polymer material. The rubbery film was heated under vacuum for 1 hour at 120° C. The brittle flakes were washed with 2×50 mL water and collected by filtration through a Whatman #1 paper filter. The solid was dried under vacuum overnight at 50-60° C. 7.65 g obtained. Ground up and sieved. D-298-182-A, 500 μm to 250 μm, 5.074 g, D-298-182-B, <250 um, 1.554 g.

Table 10. Examples of Hydrogels and IPNs Described Examples 3 and 4 Testing with the PC Test

Hydrogel copolymer (0.1 g) was weighed into a small vial. To that was added 5.00 ml of the Latanoprost formulation with BAK. The vial was sealed and then gently swirled to contact the liquid with the solid hydrogel. The vial was allowed to sit at room temperature for 48 hours. Then, the liquid was separated from the solids through a syringe with a filter and analyzed via HPLC to measure the amount of Latanoprost and BAK at equilibrium.

TABLE 10

| Ingredient | Vendor | Cat. No. | Lot No. |
|---|---|---|---|
| Latanoprost | BOC Sciences | N/A | BS17J12011 |
| HPβCD | Sigma Aldrich | C0926 | SLBT2669 |
| BAK | Sigma Aldrich | 12063 | BCBW4741 |
| Water (sterile) | Hyclone | SH30221.17 | AD21061281 |

The formulation for the Latanoprost solution was made by dissolution in sterile water of the formulation of Latanoprost: CDβHP (ratio 1:50 latanoprost: 50 ppm CDβCD, Mw˜1396 Sigma Product #C0926) with BAKs added (200 ppm).

Results are reported in parenthesis in Table 11 as percent Latanoprost unabsorbed and percent BAKs unabsorbed The Controls are the area counts of the solution of latanoprost prior to exposure to the hydrogel.

TABLE 11

| | AUC by HPLC | AUC (% of the | AUC (% of the |
|---|---|---|---|
| | (% of the | original | original |
| | original control) | control) | control) (BAK |
| Polymer | (latanoprost) | (BAK 12) | 14) |
| Control N/A | 1024 (100%) | 2846 (100%) | 1380 (100%) |
| AMPS/MBAM/TATZ | 978 (95.5%) | 314 (11.0%) | 119 (8.6%) |
| 7.5/82.5/10 | | | |
| (D-322-018-AW) | | | |
| AMPS/MBAM/TATZ | 989 (96.6%) | 309 (10.9%) | 125 (9.1%) |
| 7.5/77.5/15 | | | |
| (D-322-020-AW) | | | |
| AMPS/MBAM | 957 (93.5%) | 329 (11.2%) | 114 (8.3%) |
| 7.5/92.5 | | | |
| KPS 0.5 | | | |
| (D-322-022-AW) | | | |
| BioRad Beads/AMPS | 926 (90.4%) | 344 (12.1%) | 52 (3.8%) |
| 1 g/0.5 g | | | |
| 250-125 micron | | | |
| Not purified (D-322- | | | |
| 028-C-AW) | | | |
| Control | 956 (100.0%) | 2786 (100.0%) | 1327 (100.0%) |
| AMPS/MBAM | 931 (97.4%) | 359 (12.9%) | 157 (11.8%) |
| 7.5/92.5 extra KPS, | | | |
| ground, purified with | | | |
| IPA, water in soxhlet, | | | |
| sieved | | | |
| (D-322-002-AG-W) | | | |
| AMPS/MBAM/TATZ | 901 (94.2%) | 317 (11.4%) | 121 (9.1%) |
| 7.5/87.5/5.0 ground, | | | |
| purified, dried, | | | |
| sieved | | | |
| (D-322-006-AW) | | | |
| Control | 1025 (100.0%) | 2810 (100.0%) | 1365 (100.0%) |
| SEM/MBAM 7.5/92.5 | 1012 (98.7%) | 373 (13.3%) | 166 (12.2%) |
| purified (D-322-010- | | | |
| AW | | | |
| Control | 997 (100.0%) | 2887 (100.0%) | 1343 (100.0%) |
| AM/2-Sulfoethyl | 953 (95.6%) | 850 (29.4%) | 358 (26.7%) |
| MA(SEM)/MBAM | | | |
| 30/10/60 (D-298-132- | | | |
| A) | | | |
| Control N/A | 1019 (100.0%) | 2800 (100.0%) | 1340 (100.0%) |
| AMPS/MBAM | 1015 (99.6%) | 375 (13.4%) | 159 (11.9%) |
| 7.5/92.5 washed 3-4 h | | | |
| with water, air dried, | | | |
| sieved | | | |
| (D-298-190-AW) | | | |
| AMPS/MBAM | 990 (97.2%) | 698 (24.9%) | 298 (22.2%) |
| 7.5/92.5 extra KPS | | | |
| after 3 h, 500-250 | | | |
| micron, vacuum dried, | | | |
| ground | | | |
| (D-298-196-A) | | | |
| AMPS/MBAM | 1005 (98.6%) | 317 (11.3%) | 129 (9.6%) |
| 7.5/92.5 washed 3-4 h | | | |
| with water, air dried, | | | |
| sieved | | | |
| (D-298-196-AW) | | | |
| Control | 1014 (100.0%) | 2847 (100.0%) | 1328 (100.0%) |
| AMPS/MBAM | 979 (96.5%) | 275 (9.7%) | 88 (6.6%) |
| 7.5/92.5 Soxhlet ext | | | |
| (D-298-178-AW) | | | |
| PVA/PAMPS/CA | 905 (89.3%) | 166 (5.8%) | 36 (2.7%) |
| 4.8/1.2/2.4 IPN | | | |
| (D-298-182-A) | | | |
| AMPS/MBAM | 1024 (101.0%) | 353 (12.4%) | 150 (11.3%) |
| 7.5/92.5 ISP | | | |
| Soxhlet ext | | | |
| (D-298-184-AW) | | | |
| NVP/AMPS/MBAM/T | 978 (96.4%) | 597 (21.0%) | 257 (19.4%) |
| ATZ 30/10/30/30 (D- | | | |
| 298-186-A) | | | |
| Control | 1011 (100.0%) | 2750 (100.0%) | 1288 (100.0%) |
| AMPS/MBAM | 1006 (99.5%) | 295 (10.7%) | 126 (9.8%) |
| 7.5/92.5 (D-298-152- | | | |
| AW) | | | |
| Control | 1029 (100.0%) | 2953 (100.0%) | 1421 (100.0%) |
| N- | 954 (92.7%) | 293 (9.9%) | 76 (5.3%) |
| vinylpyrrolidinone/AM | | | |
| PS/MBAM 30/10/60 | | | |
| (D-298-120-AW) | | | |
| Control | 1003 (100.0%) | 2805 (100.0%) | 1354 (100.0%) |
| AA/SR351 40/60 (D- | 249 (24.8%) | 652 (23.2%) | 68 (5.0%) |
| 298-146-A) | | | |
| AA/MBAM/SR351 | 774 (77.2%) | 843 (30.1%) | 107 (7.9%) |
| 60/30/10 (D-298-148- | | | |
| A) | | | |
| Control | 1015 (100.0%) | 2762 (100.0%) | 1284 (100.0%) |
| AM/2-Sulfoethyl MA | 919 (90.5%) | 732 (26.5%) | 208 (16.2%) |
| (SEM)/MBAM | | | |
| 15/25/60 (D-298-134- | | | |
| A) | | | |
| AA/MBAM 40/60 (D- | 979 (96.5%) | 1588 (57.5%) | 770 (60.0%) |
| 298-140-A) | | | |
| AA/MBAM 50/50 (D- | 953 (93.9%) | 1330 (48.2%) | 645 (50.2%) |
| 298-142-A) | | | |
| VP/AA/MBAM | 973 (95.9%) | 1228 (44.5%) | 606 (47.2%) |
| 10/45/45 (D-298-144- | | | |
| A) | | | |

### Example 5

Two Experimental Hydrogels Testing: Example with CDβHP and Example without CDβHP:

### Partition Coefficient (PC) Test:

Hydrogel copolymer (0.1 g) was weighed into a small vial. To that was added 5.00 ml of the Latanoprost formulation with BAK. The vial was sealed and then gently swirled to contact the liquid with the solid hydrogel. The vial was allowed to sit at room temperature for 48 hours. Then, the liquid was separated from the solids through a syringe with a filter and analyzed via HPLC to measure the amount of Latanoprost and BAK at equilibrium.

TABLE 12

| Ingredient | Vendor | Cat. No. | Lot No. |
|---|---|---|---|
| Latanoprost | BOC Sciences | N/A | BS17J12011 |
| HPβCD | Sigma Aldrich | C0926 | SLBT2669 |
| BAK | Sigma Aldrich | 12063 | BCBW4741 |
| Water (sterile) | Hyclone | SH30221.17 | AD21061281 |

The formulation for the Latanoprost solution was made by dissolution in sterile water of the formulation of Latanoprost:CDβHP (ratio 1:50 latanoprost: 50 ppm CDβCD, Mw˜1396 Sigma Product #C0926) with BAKs added (200 ppm).

Results are reported as percent Latanoprost unabsorbed and percent BAKs absorbed. Or as percent Latanoprost absorbed and percent BAKs absorbed.

### Results:

A partition coefficient (PC) test with latanoprost formulation with and without CD: The Control formulation of latanoprost (50 ppm) with BAK (200 ppm) in water (sterile, Hyclone Product #SH30221.17) was prepared via dissolution (formulation pH 6.6). The partition coefficient tests with hydrogels (500-250 micron) were carried out for 48 h. The results were shown in the table and graphic below.

The Experimental inventive formulation of Latanoprost:CD (ratio 1:50 latanoprost: 50 ppm, Mw˜1396 Sigma Product #C0926) with BAK (200 ppm) in water was prepared via dissolution (formulation pH 8.4). The partition coefficient tests were carried out for 48 hr and the results were shown in the table below. Here are reported % latanoprost unabsorbed and % BAK absorbed.

TABLE 13

| | % latanoprost | % BAK 12 | % BAK 14 |
|---|---|---|---|
| Polymer Matrix | unabsorbed | absorbed | absorbed |
| D-298-120-AW2 with CD | 93 | 90 | 95 |
| D-298-152-AW with CD | 100 | 91 | 90 |
| D-298-120-AW2 without CD | 40 | 100 | 100 |
| D-298-152-AW without CD | 35 | 100 | 100 |

The presence of CD reduces absorption of the Latanoprost (% unabsorbed >90%) and yet still absorb greater than 90% of the BAKs in this screening experiment. In some cases, the use of these types of hydrophilic copolymer hydrogels with anionic functionality will absorb most or all of the preservative such as BAK. However, the complexing agent may be beneficial to keep the ophthalmic agent (e.g., latanoprost) soluble and unabsorbed by the hydrogel.

### Example 6: Dropping Bottle Testing of the Hydrogel

The results are for 5 bottle tips prepared with hydrogel D-298-152 AW described above. Solutions as described above with Latanoprost, CD and BAKs at the concentrations described here. The Experimental formulation of mole ratio of Latanoprost:CD (mole ratio 1:50); latanoprost concentration: 50 ppm, HPβCD used was Mw˜1396 Sigma Product #C0926) with BAK used from Sigma product #12063 (200 ppm) in water was prepared via dissolution as described previously.

Over 30 days, 2 drops/day of solution were collected from each of the 5 bottles and analyzed for Latanoprost and BAK via HPLC.

Results show the Latanoprost in the collected drops is >95% of the initial 50 ppm in the bottle originally and that nearly all the BAK was absorbed with some breakthrough toward the end of the 30 days in several of the bottles.

TABLE 14
Latanoprost (μg/mL)

| Day | Bottle #1 | Bottle #2 | Bottle #3 | Bottle #4 | Bottle #5 |
|---|---|---|---|---|---|
| 1 | 52.7 | 54.7 | 49.0 | 50.9 | 52.5 |
| 2 | 50.7 | 50.0 | 48.9 | 49.9 | 51.1 |
| 3 | 51.8 | 54.1 | 51.5 | 49.7 | 51.9 |
| 4 | 53.1 | 50.1 | 50.2 | 50.6 | 52.2 |
| 5 | 52.4 | 53.1 | 50.8 | 54.8 | 56.4 |
| 6 | 52.6 | 52.4 | 50.4 | 51.5 | 52.1 |
| 7 | 53.1 | 49.3 | 49.5 | 51.4 | 51.3 |
| 8 | 52.9 | 52.5 | 48.1 | 45.4 | 52.2 |
| 9 | 52.1 | 52.8 | 53.2 | 48.9 | 50.7 |
| 10 | 51.5 | 49.6 | 53.8 | 53.6 | 52.1 |
| 11 | 51.2 | 48.9 | 50.3 | 48.1 | 45.5 |
| 12 | 50.3 | 51.8 | 52.5 | 47.1 | 51.8 |
| 13 | 52.5 | 50.2 | 46.6 | 49.7 | 48.9 |
| 14 | 49.4 | 49.2 | 51.9 | 48.1 | 51.9 |
| 15 | 49.4 | 51.4 | 48.8 | 47.3 | 50.0 |
| 16 | 48.7 | 49.2 | 47.7 | 48.2 | 47.3 |
| 17 | 48.6 | 50.3 | 44.7 | 47.5 | 48.5 |
| 18 | 50.4 | 48.4 | 46.5 | 47.0 | 47.8 |
| 19 | 49.4 | 49.4 | 49.5 | 47.0 | 47.7 |
| 20 | 49.6 | 50.0 | 47.7 | 47.3 | 46.6 |
| 21 | 49.1 | 51.0 | 50.4 | 47.4 | 47.3 |
| 22 | 48.7 | 48.2 | 49.5 | 48.1 | 48.2 |
| 23 | 50.1 | 49.9 | 50.5 | 48.6 | 48.7 |
| 24 | 48.4 | 50.8 | 49.7 | 49.2 | 48.2 |
| 25 | 48.8 | 48.5 | 49.6 | 48.6 | 49.7 |
| 26 | 40.5 | 38.2 | 44.3 | 43.3 | 45.5 |
| 27 | 48.8 | 49.4 | 49.0 | 47.9 | 47.5 |
| 28 | 48.1 | 48.7 | 49.8 | 47.6 | 48.3 |
| 29 | 48.5 | 49.2 | 49.0 | 47.7 | 47.6 |
| 30 | 48.8 | 48.2 | 49.2 | 47.3 | 48.9 |

TABLE 15
BAK (μg/mL)

| Day | Bottle #1 | Bottle #2 | Bottle #3 | Bottle #4 | Bottle #5 |
|---|---|---|---|---|---|
| 1 | ND | 1.54 | 0.327 | 13.5 | 5.80 |
| 2 | ND | 0.294 | ND | 0.575 | 2.33 |
| 3 | ND | 0.512 | 0.443 | 0.554 | 1.15 |
| 4 | ND | 0.312 | ND | 0.278 | 1.36 |
| 5 | ND | 0.508 | 0.317 | 0.441 | 1.49 |
| 6 | ND | 0.539 | 0.133 | 0.720 | 1.10 |
| 7 | ND | 0.542 | 0.182 | 0.805 | 1.63 |
| 8 | ND | 0.525 | ND | 0.808 | 1.28 |
| 9 | ND | 0.941 | 0.477 | 0.779 | 1.83 |
| 10 | ND | 0.830 | 0.842 | 0.741 | 1.70 |
| 11 | ND | 1.36 | 1.17 | 0.844 | 1.91 |
| 12 | ND | 1.07 | 0.507 | 0.968 | 2.16 |
| 13 | ND | 1.57 | 1.14 | 0.834 | 2.00 |
| 14 | ND | 1.73 | 1.64 | 1.08 | 2.67 |
| 15 | ND | 1.28 | 2.24 | 1.25 | 2.67 |
| 16 | ND | 1.89 | 2.78 | 1.42 | 2.62 |
| 17 | 0.245 | 1.90 | 2.08 | 1.47 | 2.82 |
| 18 | 0.269 | 2.15 | 2.04 | 1.62 | 2.85 |
| 19 | 0.499 | 2.22 | 3.21 | 1.75 | 3.32 |
| 20 | 0.638 | 2.68 | 1.97 | 2.04 | 3.85 |
| 21 | 0.580 | 2.79 | 2.24 | 2.10 | 4.11 |
| 22 | 0.453 | 2.13 | 2.29 | 2.48 | 3.86 |
| 23 | 0.574 | 2.69 | 2.86 | 2.56 | 4.35 |
| 24 | 0.747 | 2.99 | 3.91 | 2.83 | 4.26 |
| 25 | 0.680 | 3.43 | 4.42 | 2.94 | 4.51 |
| 26 | 0.635 | 1.69 | 3.44 | 2.88 | 4.50 |
| 27 | 0.889 | 3.05 | 5.14 | 2.77 | 5.14 |
| 28 | 1.11 | 4.61 | 4.17 | 3.24 | 5.79 |
| 29 | 1.13 | 4.30 | 4.89 | 3.67 | 7.01 |
| 30 | 1.18 | 3.74 | 5.22 | 4.07 | 7.15 |

### Example 7

Bio Gel P Beads Modified with Sulfoethylmethacrylate (SEM)

Bio Gel P-4 (90-180 micron size) beads were purchased directly from Bio-Rad Corporation of Hercules Calif. Bio-Gel P gels are porous polyacrylamide beads prepared by copolymerization of acrylamide and N,N′-methylene-bis-acrylamide (A/C type monomers). The beads are extremely hydrophilic and essentially free of charge, and provide efficient, gentle gel filtration of sensitive compounds. Their synthetic composition and freedom from soluble impurities preclude eluate contamination. High resolution is assured by consistent narrow distribution of bead diameters and excellent molecular weight discrimination These were used in the examples without further purification.

D-322-034 Addition of SEM (B type monomer) to the P-4 Bio-Rad beads, modification of crosslinked polyacrylamide beads by SEM. So-called “grafting” polymerization.

To a solution of SEM (2-Sulfoethyl Methacrylate) in water was added the beads Bio-Gel P-4 Gel, Medium, and the mixture was heated to 55° C. Then KPS (2 mol %, 40 mg/mL stock solution in water) was added to the slurry of beads and SEM. The temperature was increased to 70° C. for 6 hours. The product was worked-up by centrifuge washing with IPA and water in a 50 mL tube. The solid was collected by filtration and dried in a vacuum oven. The dried solid was sieved, purified with water and then with IPA in a soxhlet extractor, and dried. Finally, the dried product was re-sieved to get particles between 500 and 250 microns mainly.

TABLE 16
Charge Table per 20 mL vial

| Cmpd | D-322-034-02 | D-322-034-03 |
|---|---|---|
| SEM | 0.5 g | 0.9408 g |
| water | 10 mL | 10 mL |
| | (20 vol) | (10 vol) |
| Bio-Rad P-4 | 1.0 g | 0.94 g |
| beads | | |
| KPS | 26.1 mg | 27.8 mg |
| | (0.35 mL) | (0.70 mL) |

SEM = Sulfoethyl Methacrylate

### D-322-034-02-A 500 μm to 250 μm, 0.2667 g

### D-322-034-03-AW 500 μm to 250 μm, 0.2659 g

PC tests of the hydrogels: the formulation of latanoprost/CD ( 1/50, latanoprost: 50 ppm, HPβCD Mw˜1396 Sigma Product #C0926) with BAK (200 ppm, Sigma Product #12063) in water was prepared via dissolution (formulation pH 8.1). The partition coefficient tests with the specified hydrogels (100 mg each) were carried out for 48 h in 5 mL of the above formulation and the results were shown below when analyzed by HPLC. The filtration of D-322-034-02-AW and D-322-034-03-AW was similar to the unmodified BioRad beads. No impurities were found at the solvent front of D-322-034-02-AW and D-322-034-03-AW. Hydrogel D-322-034-02-AW and D-322-034-03-AW showed very low absorption of latanoprost and very high absorption of BAK.

TABLE 17

| | | AUC (% of the | AUC (% of the | AUC (% of the |
|---|---|---|---|---|
| | | original control) | original control) | original control) |
| | | at ~2.7 min | at ~3.8 min | at ~4.8 min |
| Exp | Polymer | (latanoprost) | (BAK 12) | (BAK 14) |
| control | N/A | 984 (100%) | 2747 (100%) | 1314 (100%) |
| D-322- | SEM/BioRad | 981 (99.7%) | 69 (2.5%) | 8 (0.6%) |
| 034-02- | Beads 50/50, | | | |
| AW | water (20 | | | |
| | vol.), 500- | | | |
| | 250 micron, | | | |
| D-322- | SEM/BioRad | 977 (99.3%) | 61 (2.2%) | 7 (0.5%) |
| 034-03- | Beads 50/50, | | | |
| AW | water (10 | | | |
| | vol.), 500- | | | |
| | 250 micron, | | | |

### Comparative Example 8: Control with as Received Bio-Rad Beads: PC Test and Tip Flow Test with the Bio-Rad, Bio-Gel P-4, Medium Sized Beads (90-180 μm), (No Modification, Used as Received from Bio-Rad Corporation)

The formulation of latanoprost/CD ( 1/50, latanoprost: 50 ppm, HPβCD Mw˜1396 Sigma Product #C0926) with BAK (200 ppm, Sigma Product #12063) in water was prepared via dissolution (formulation pH 8.6). The partition coefficient tests with BioRad beads (Bio-Gel P-4 Media Cat #150-4120, 100 mg, 180-90 micron) were carried out for 48 h in 5 mL of the above formulation (Table 16). The results were shown in the table below. The BioRad beads that have not been modified “grafted” with SEM showed poor absorption for BAK compared to the SEM modified, “grafted” beads such as D-322-034-02-AW and D-322-034-03-AW. Hydrogels shown in Table 17.

TABLE 18

| | | AUC (% of | AUC (% of | AUC (% of |
|---|---|---|---|---|
| | | the original) | the original) | the original) |
| | | at ~2.7 min | at ~3.8 min | at ~4.8 min |
| Exp | Polymer | (latanoprost) | (BAK 12) | (BAK 14) |
| control | N/A | 925 (100.0%) | 2770 (100.0%) | 1348 (100.0%) |
| Bio-Gel | BioRad Bio- | 938 (101.4%) | 2144 (77.4%) | 1034 (76.7%) |
| P-4 | Gel P-4 Media | | | |
| | Cat #150- | | | |
| | 4120, 180-90 | | | |
| | micron | | | |

### Example 9

30 Drop Testing in Bottles with Hydrogel Packed Tips

Procedure: The results are for 5 bottle tips prepared with hydrogels described above. Molded plastic tips were packed with purified hydrogel. Exp 26 (a1-a3) (˜100 mg in each tip) hydrogel: SEM/MBAM 10/90, 500-250 micron, D-322-040-AW. 26 (b1-b3) (˜100 mg packed in each tip) hydrogel: SEM/BioRad P-4, 500-250 micron, D-322-056-02-AW: the formulation was able to be squeezed through the tip to form drops at the tip for collection.

The formulation placed in each of the 6 bottles was prepared as described previously with latanoprost/CD ( 1/50, latanoprost: 50 ppm, HPβCD Mw˜1396 Sigma Product #C0926) with BAK (100 ppm) in water was prepared (pH 8.27) and 3 mL were added to each bottle.

The hydrogel (copolymer matrix) mixture in the tips was soaked with 400 μL of the above formulation followed by closing with the back filter on the tip and fixing each tip to each bottle. The bottles were invert and squeezing of the bottle was performed such that the formulation passed through the polymer matrix in the tips. Around two drops were taken at each time (30-50 μL/drop) followed by dilution with acetonitrile. The resulting mixture was subjected to HPLC analysis with a C8 guard column to filter the small particles. The HPLC results could be used to measure the original concentration of latanoprost and BAKs in the bottle at 50 ppm and 100 ppm. The results of the drop testing analysis are shown Tables 17 and 18 below.

No BAK was identified or detected in the any of the drops collected during the experiment. The latanoprost was measure at ˜50 ppm in the bottle and in the drops collected for all 30 days of the experiment.

TABLE 19

| Day | | | | | | | ppm/ | | | | | |
|---|---|---|---|---|---|---|---|---|---|---|---|---|
| Number (2 | Area under the curve | | | | | | Latanoprost | | | | | |
| drops per | data from HPLC | | | | | | in | | | | | |
| sample/day) | 26a1 | | | | | | drops | | | | | |
| (in bottle) | 1081 | 26a2 | 26a3 | 26b1 | 26b2 | 26b3 | 26a1 | 26a2 | 26a3 | 26b1 | 26b2 | 26b3 |
| 1 | 1017 | 1033 | 986 | 1072 | 1113 | 1101 | 47.0 | 47.8 | 45.6 | 49.6 | 51.5 | 50.9 |
| 2 | 1069 | 1136 | 1058 | 1094 | 1080 | 1085 | 49.4 | 52.5 | 48.9 | 50.6 | 50.0 | 50.2 |
| 3 | 1029 | 1041 | 1005 | 1074 | 1073 | 1077 | 47.6 | 48.1 | 46.5 | 49.7 | 49.6 | 49.8 |
| 4 | 1097 | 1102 | 1065 | 1047 | 1108 | 1071 | 50.7 | 51.0 | 49.3 | 48.4 | 51.2 | 49.5 |
| 5 | 1083 | 1078 | 1064 | 1057 | 1122 | 1077 | 50.1 | 49.9 | 49.2 | 48.9 | 51.9 | 49.8 |
| 6 | 1071 | 1114 | 1078 | 1047 | 1102 | 1087 | 49.5 | 51.5 | 49.9 | 48.4 | 51.0 | 50.3 |
| 7 | 1034 | 1086 | 1041 | 1134 | 1068 | 1072 | 47.8 | 50.2 | 48.1 | 52.5 | 49.4 | 49.6 |
| 8 | 1033 | 1026 | 1026 | 1109 | 1039 | 1081 | 47.8 | 47.5 | 47.5 | 51.3 | 48.1 | 50.0 |
| 9 | 1017 | 1031 | 1037 | 1030 | 1069 | 1064 | 47.0 | 47.7 | 48.0 | 47.6 | 49.4 | 49.2 |
| 10 | 1035 | 1035 | 1044 | 1026 | 1035 | 1048 | 47.9 | 47.9 | 48.3 | 47.5 | 47.9 | 48.5 |
| 11 | 1017 | 1015 | 1020 | 1023 | 1082 | 1049 | 47.0 | 46.9 | 47.2 | 47.3 | 50.0 | 48.5 |
| 12 | 1030 | 1026 | 1041 | 1035 | 1062 | 1048 | 47.6 | 47.5 | 48.1 | 47.9 | 49.1 | 48.5 |
| 13 | 1040 | 1020 | 1034 | 1060 | 1085 | 1055 | 48.1 | 47.2 | 47.8 | 49.0 | 50.2 | 48.8 |
| 14 | 1018 | 1032 | 1047 | 1060 | 1040 | 1091 | 47.1 | 47.7 | 48.4 | 49.0 | 48.1 | 50.5 |
| 15 | 1035 | 1025 | 1045 | 1090 | 1097 | 1065 | 47.9 | 47.4 | 48.3 | 50.4 | 50.7 | 49.3 |
| 16 | 1060 | 1007 | 1038 | 1026 | 1029 | 1074 | 49.0 | 46.6 | 48.0 | 47.5 | 47.6 | 49.7 |
| 17 | 1055 | 1009 | 1049 | 1060 | 1019 | 1047 | 48.8 | 46.7 | 48.5 | 49.0 | 47.1 | 48.4 |
| 18 | 1041 | 1027 | 1038 | 1024 | 1154 | 1036 | 48.1 | 47.5 | 48.0 | 47.4 | 53.4 | 47.9 |
| 19 | 1016 | 1009 | 1042 | 1015 | 1036 | 1047 | 47.0 | 46.7 | 48.2 | 46.9 | 47.9 | 48.4 |
| 20 | 1024 | 1007 | 1039 | 1043 | 1107 | 1076 | 47.4 | 46.6 | 48.1 | 48.2 | 51.2 | 49.8 |
| 21 | 1027 | 998 | 1031 | 1030 | 1039 | 1072 | 47.5 | 46.2 | 47.7 | 47.6 | 48.1 | 49.6 |
| 22 | 1003 | 988 | 1018 | 1085 | 1037 | 1064 | 46.4 | 45.7 | 47.1 | 50.2 | 48.0 | 49.2 |
| 23 | 1032 | 1004 | 1043 | 1029 | 1039 | 1052 | 47.7 | 46.4 | 48.2 | 47.6 | 48.1 | 48.7 |
| 24 | 1027 | 1030 | 1046 | 1045 | 1093 | 1056 | 47.5 | 47.6 | 48.4 | 48.3 | 50.6 | 48.8 |
| 25 | 1052 | 1008 | 1036 | 1071 | 1068 | 1065 | 48.7 | 46.6 | 47.9 | 49.5 | 49.4 | 49.3 |
| 26 | 1027 | 1014 | 1043 | 1026 | 1053 | 1065 | 47.5 | 46.9 | 48.2 | 47.5 | 48.7 | 49.3 |
| 27 | 1017 | 998 | 1036 | 1024 | 1056 | 1054 | 47.0 | 46.2 | 47.9 | 47.4 | 48.8 | 48.8 |
| 28 | 1025 | 1021 | 1042 | 1038 | 1047 | 1072 | 47.4 | 47.2 | 48.2 | 48.0 | 48.4 | 49.6 |
| 29 | 1027 | 999 | 1026 | 1020 | 1057 | 1064 | 47.5 | 46.2 | 47.5 | 47.2 | 48.9 | 49.2 |
| 30 | 1013 | 1009 | 1045 | 1010 | 1032 | 1067 | 46.9 | 46.7 | 48.3 | 46.7 | 47.7 | 49.4 |

TABLE 20

| | | Total BAKs (C12 and C14) Measured | | | | |
|---|---|---|---|---|---|---|
| Day Number (2 | | (ND = Non-Detectable = <0.1 ppm) | | | | |
| drops/sample/day) | 26a1 | 26a2 | 26a3 | 26b1 | 26b2 | 26b3 |
| 0 | | | | | | |
| 1 | ND | ND | ND | ND | ND | ND |
| 2 | ND | ND | ND | ND | ND | ND |
| 3 | ND | ND | ND | ND | ND | ND |
| 4 | ND | ND | ND | ND | ND | ND |
| 5 | ND | ND | ND | ND | ND | ND |
| 6 | ND | ND | ND | ND | ND | ND |
| 7 | ND | ND | ND | ND | ND | ND |
| 8 | ND | ND | ND | ND | ND | ND |
| 9 | ND | ND | ND | ND | ND | ND |
| 10 | ND | ND | ND | ND | ND | ND |
| 11 | ND | ND | ND | ND | ND | ND |
| 12 | ND | ND | ND | ND | ND | ND |
| 13 | ND | ND | ND | ND | ND | ND |
| 14 | ND | ND | ND | ND | ND | ND |
| 15 | ND | ND | ND | ND | ND | ND |
| 16 | ND | ND | ND | ND | ND | ND |
| 17 | ND | ND | ND | ND | ND | ND |
| 18 | ND | ND | ND | ND | ND | ND |
| 19 | ND | ND | ND | ND | ND | ND |
| 20 | ND | ND | ND | ND | ND | ND |
| 21 | ND | ND | ND | ND | ND | ND |
| 22 | ND | ND | ND | ND | ND | ND |
| 23 | ND | ND | ND | ND | ND | ND |
| 24 | ND | ND | ND | ND | ND | ND |
| 25 | ND | ND | ND | ND | ND | ND |
| 26 | ND | ND | ND | ND | ND | ND |
| 27 | ND | ND | ND | ND | ND | ND |
| 28 | ND | ND | ND | ND | ND | ND |
| 29 | ND | ND | ND | ND | ND | ND |
| 30 | ND | ND | ND | ND | ND | ND |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for administering an ophthalmic agent, comprising:
providing a solution, emulsion, or suspension comprising a hydrophobic ophthalmic agent, a preservative, and a complexing agent, wherein the complexing agent is configured to form a complex with the hydrophobic ophthalmic agent; and
providing a polymeric matrix, wherein the complexing agent is configured to reduce an affinity of the ophthalmic agent for the polymeric matrix and wherein the polymeric matrix is configured to selectively absorb the preservative when the solution, emulsion, or suspension is passed therethrough.

2. The method of claim 1, wherein the complexing agent and the hydrophobic ophthalmic agent form an inclusion compound.

3. The method of claim 2, wherein the complexing agent comprises a cyclodextrin.

4. The method of claim 3, wherein the cyclodextrin is sized to host the hydrophobic ophthalmic agent within a hydrophobic interior of the cyclodextrin.

5. The method of claim 3, wherein the cyclodextrin is at least one of (2-Hydroxypropyl)-α-cyclodextrin, (2-Hydroxypropyl)-β-cyclodextrin, (2-Hydroxypropyl)-γ-cyclodextrin, α-cyclodextrin, β-cyclodextrin, γ-cyclodextrin, methyl-α-cyclodextrin, methyl-β-cyclodextrin, methyl-γ-cyclodextrin, dimethyl-beta-cyclodextrin, highly sulphated-beta-cyclodextrin, 6-monodeoxy-6-N-mono(3-hydroxy)propylamino-beta-cyclodextrin, or a randomly or selectively substituted alpha, beta or gamma cyclodextrin.

6. The method of claim 1, wherein a concentration of the complexing agent is less than 200 micromolar.

7. The method of claim 1, wherein a concentration of the complexing agent is greater than the concentration of the ophthalmic agent by about 10:1 by mole to about 200:1 by mole.

8. The method of claim 7, wherein a concentration of the complexing agent is greater than the concentration of the ophthalmic agent by at least 2 percent by mole.

9. The method of claim 1, wherein the complexing agent is a micelle forming surfactant.

10. The method of claim 1, wherein the hydrophobic ophthalmic agent comprises latanoprost, bimatoprost, dexamethasone, cyclosporine or travoprost, or any prostaglandin analog drug.

11. The method of claim 1, wherein the concentration of the ophthalmic agent is less than 200 millimolar.

12. The method of claim 1, wherein the concentration of the ophthalmic agent is less than 0.05% by weight.

13. The method of claim 1, wherein the preservative is benzalkonium chloride.

14. The method of claim 1, where the concentration of the preservative is less than 0.05% by weight.

15. The method of claim 1, wherein the polymeric matrix is a polymeric hydrogel.

16. The method of claim 1, wherein the polymeric matrix comprises 2-hydroxyethylmethacrylate.

17. The method of claim 1, wherein the polymeric matrix comprises tert-butyl methacrylate.

18. The method of claim 1, wherein the polymeric matrix comprises a crosslinker.

19. The method of claim 18, wherein the crosslinker is SR-9035.

20. The method of claim 1, wherein the solution, emulsion, or suspension is disposed within a chamber of a compressible bottle.

21. The method of claim 1, wherein the concentration of the ophthalmic agent after passing though the polymeric matrix is at least 80% of a concentration of the ophthalmic agent before passing through the polymeric matrix.

22. The method of claim 1, wherein the concentration of the preservative after passing though the polymeric matrix is less than 10% of the concentration of the preservative before passing through the polymeric matrix.

23. The method of claim 1, wherein the polymeric matrix comprises polyvinyl alcohol crosslinked with a crosslinking agent to render it a hydrogel.

24. The method of claim 1, wherein the polymeric matrix is selected from crosslinked polyvinylpyrrolidone, crosslinked polyethylene oxide, crosslinked polyacrylamides, crosslinked copolymers of methacrylic acid, polyacrylic acid, or copolymers selected from poly (acrylic acid-co-acrylamide), or poly (methacrylic acid-co-acrylamide).

25. The method of claim 1, wherein the polymeric matrix is hydrogel prepared from polyacrylamide crosslinked with at least one crosslinking monomer selected from N,N′-methylenebis(acrylamide) (MBAM), triacrylamido triazine (TATZ), SR 351, or SR9035; and the crosslinked polyacrylamide is modified with at least one modifying monomer selected from methyl acrylate (MAA), 2-acrylamido-2-methylpropane sulfonic acid (AMPS), 2-sulfoethyl methacrylate (SEM), acrylic acid (AA), or vinylphosphonic acid (VP).

26. The method of claim 1, wherein the polymeric matrix is hydrogel prepared from polyacrylamide crosslinked with N,N′-methylenebis(acrylamide) (MBAM); and the crosslinked polyacrylamide is modified with 2-sulfoethyl methacrylate (SEM).

27. The method of claim 1, wherein the polymeric matrix is hydrogel prepared from polyacrylamide crosslinked with at least one crosslinking monomer selected from N,N′-methylenebis(acrylamide) (MBAM), triacrylamido triazine (TATZ), SR 351, or SR9035; the crosslinked polyacrylamide material is isolated; and the crosslinked polyacrylamide material is modified with at least one modifying monomer selected from methyl acrylate (MAA), 2-acrylamido-2-methylpropane sulfonic acid (AMPS), 2-sulfoethyl methacrylate (SEM), acrylic acid (AA), or vinylphosphonic acid (VP).

28. The method of claim 1, wherein the polymeric matrix is hydrogel prepared from polyacrylamide crosslinked with N,N′-methylenebis(acrylamide) (MBAM); the crosslinked polyacrylamide material is isolated; and the crosslinked polyacrylamide material is modified with at least one modifying monomer selected from 2-acrylamido-2-methylpropane sulfonic acid (AMPS), or 2-sulfoethyl methacrylate (SEM).

29. The method of claim 1, wherein the crosslinked polyacrylamide material is isolated in the form of spherical beads.
